SEQUENCE LISTING (86 sequences)

SEQ ID NO: 1 - DNA, 81 bp, Artificial sequence
  synthetic sequence
aggtctatataagcagagctcgtttagtgaaccgtcagatcgcctggagacgtcgagccgagtggttgtgcctccatagaa

SEQ ID NO: 2 - DNA, 81 bp, Artificial sequence
  synthetic sequence
gtacttatataagggggtgggggcgcgttcgtcctcagtcgcgatcgaacactcgagccgagcagacgtgcctacggaccg

SEQ ID NO: 3 - DNA, 81 bp, Artificial sequence
  synthetic sequence
aggtctatataagcagagctcgtttagtgaaccgtcagatcgcctggagacgccatccacgctgttttgacctccatagaa

SEQ ID NO: 4 - DNA, 100 bp, Artificial sequence
  synthetic sequence
aggaggtgggggacccagaggggctttgacgtcagcctggcctttaagaggccgcctgcctggcaagggctgtggagacagaactcgggaccaccagctt

SEQ ID NO: 5 - DNA, 108 bp, Artificial sequence
  synthetic sequence
atgacatcatcttcaaatgctgagtcatcaaacccccgcccccgcccaaatgggcgtggccaaactcagccaatcagcgcaaaaccccgcccccaaatattgcacaat

SEQ ID NO: 6 - DNA, 108 bp, Artificial sequence
  synthetic sequence
gttgacctttgacctttcaaaaatatgcaaataacaaagcacgtgcaaaattgcatcatcccaaaatgagtcacacaaaatgacatcatcttcaaaattgcatcatcc

SEQ ID NO: 7 - DNA, 116 bp, Artificial sequence
  synthetic sequence
atgactcagcacaaatgacgtcacaaatattgcacaatcaaaatgagtcacacaaaaccccgcccccaaaattgcatcatcccaaaatgacatcatcttcaaattatttgcatatt

SEQ ID NO: 8 - DNA, 128 bp, Artificial sequence
  synthetic sequence
caaagtaaacatggacaaaattgtttacgtttgcaaaatgtttaccaaatccttgacctttgcaaaccggaagtggccaaatacgcccacgcattcaaatacgcccacgcattcaaaccggaagtggc

SEQ ID NO: 9 - DNA, 133 bp, Artificial sequence
  synthetic sequence
tacgcccacgcattcaaaagttaatcattaactcaaatgcgcgtgcgcacaaatttggcgccaaacaaaggtgacgtcacccaaatgctgagtcatcaaacaaacgtaaacaatcaaagtataaaaggcgggg

SEQ ID NO: 10 - DNA, 90 bp, Artificial sequence
  synthetic sequence
tccttgacctttgcaaaatgactcagcacaaaatgactcagcacaaatccttgacctttgcaaaatgactcagcacaaatgctgagtcat

SEQ ID NO: 11 - DNA, 193 bp, Artificial sequence
  synthetic sequence
gttgacctttgacctttcaaaaatatgcaaataacaaagcacgtgcaaaattgcatcatcccaaaatgagtcacacaaaatgacatcatcttcaaaattgcatcatcccaaaaggtctatataagcagagctcgtttagtgaaccgtcagatcgcctggagacgtcgagccgagtggttgtgcctccatagaa

SEQ ID NO: 12 - DNA, 218 bp, Artificial sequence
  synthetic sequence
tacgcccacgcattcaaaagttaatcattaactcaaatgcgcgtgcgcacaaatttggcgccaaacaaaggtgacgtcacccaaatgctgagtcatcaaacaaacgtaaacaatcaaagtataaaaggcggggcaaaaggtctatataagcagagctcgtttagtgaaccgtcagatcgcctggagacgtcgagccgagtggttgtgcctccatagaa

SEQ ID NO: 13 - DNA, 69 bp, Artificial sequence
  synthetic sequence
ttacctgtggaagcaaatacttccttcaaagagtcttcaaagaagacacactctgttactcgcctcgac

SEQ ID NO: 14 - DNA, 75 bp, Artificial sequence
  synthetic sequence
ttacctgtggaagcaaactcagccaatcagcgcaaagagtcttcaaagaagacacactcatttactcgcctcgac

SEQ ID NO: 15 - DNA, 86 bp, Artificial sequence
  synthetic sequence
ttacctgtggaagcaaatacttccttcaaaacttcctcaaaggagaccaaaggtctccactcactgactctgttactcgcctcgac

SEQ ID NO: 16 - DNA, 86 bp, Artificial sequence
  synthetic sequence
ttacctgtggaagcaaaaggaagtcaaaaaggaagtacaaaggagaccaaaggtctccactcaacaactccagtactcgcctcgac

SEQ ID NO: 17 - DNA, 92 bp, Artificial sequence
  synthetic sequence
ttacctgtggaagcaaaaggaagtcaaacgctgattggctgagcaaaggagaccaaaggtctccactcaaatactccagtactcgcctcgac

SEQ ID NO: 18 - DNA, 94 bp, Artificial sequence
  synthetic sequence
ttacctgtggaagcaaatacttccttcaaacgctgattggctgagcaaaggagaccaaaggtctccactcaaatactctgttactcgcctcgac

SEQ ID NO: 19 - DNA, 93 bp, Artificial sequence
  synthetic sequence
tacctgtggaagcaaactcagccaatcagcgcaaaaaggaagtacaaaggagaccaaaggtctccactcaacaactcatttactcgcctcgac

SEQ ID NO: 20 - DNA, 107 bp, Artificial sequence
  synthetic sequence
ttacctgtggaagcaaatacttccttcaaaacttcctcaaaaggaagtcaaagagtcttcaaagaagacacactccagtactcactgactctgttactcgcctcgac

SEQ ID NO: 21 - DNA, 109 bp, Artificial sequence
  synthetic sequence
ttacctgtggaagcaaatacttccttcaaaaaggaagtacaaaaggaagtcaaagagtcttcaaagaagacacactccagtactcaacaactctgttactcgcctcgac

SEQ ID NO: 22 - DNA, 115 bp, Artificial sequence
  synthetic sequence
ttacctgtggaagcaaactcagccaatcagcgcaaaacttcctcaaatacttccttcaaagagtcttcaaagaagacacactctgttactcactgactcatttactcgcctcgac

SEQ ID NO: 23 - DNA, 115 bp, Artificial sequence
  synthetic sequence
ttacctgtggaagcaaaaggaagtcaaacgctgattggctgagcaaatacttccttcaaagagtcttcaaagaagacacactctgttactcaaatactccagtactcgcctcgac

SEQ ID NO: 24 - DNA, 109 bp, Artificial sequence
  synthetic sequence
ttacctgtggaagcaaaaggaagtcaaaaaggaagtacaaatacttccttcaaagagtcttcaaagaagacacactctgttactcaacaactccagtactcgcctcgac

SEQ ID NO: 25 - DNA, 88 bp, Artificial sequence
  synthetic sequence
ttacctgtggaagcaaatacttccttcaaaaaggaagtacaaaggagaccaaaggtctccactcaacaactctgttactcgcctcgac

SEQ ID NO: 26 - DNA, 94 bp, Artificial sequence
  synthetic sequence
ttacctgtggaagcaaactcagccaatcagcgcaaaaaggaagtacaaaggagaccaaaggtctccactcaacaactcatttactcgcctcgac

SEQ ID NO: 27 - DNA, 67 bp, Artificial sequence
  synthetic sequence
ttacctgtggaagcaaaaggaagtcaaagagtcttcaaagaagacacactccagtactcgcctcgac

SEQ ID NO: 28 - DNA, 115 bp, Artificial sequence
  synthetic sequence
ttacctgtggaagcaaatacttccttcaaaacttcctcaaactcagccaatcagcgcaaagagtcttcaaagaagacacactcatttactcactgactctgttactcgcctcgac

SEQ ID NO: 29 - DNA, 113 bp, Artificial sequence
  synthetic sequence
ttacctgtggaagcaaactcagccaatcagcgcaaaacttcctcaaaaggaagtcaaagagtcttcaaagaagacacactccagtactcactgactcatttactcgcctcgac

SEQ ID NO: 30 - DNA, 111 bp, Artificial sequence
  synthetic sequence
ttacctgtggaagcaaatacttccttcaaaaaggaagtacaaatacttccttcaaagagtcttcaaagaagacacactctgttactcaacaactctgttactcgcctcgac

SEQ ID NO: 31 - DNA, 107 bp, Artificial sequence
  synthetic sequence
ttacctgtggaagcaaaaggaagtcaaaacttcctcaaatacttccttcaaagagtcttcaaagaagacacactctgttactcactgactccagtactcgcctcgac

SEQ ID NO: 32 - DNA, 109 bp, Artificial sequence
  synthetic sequence
ttacctgtggaagcaaatacttccttcaaaacttcctcaaatacttccttcaaagagtcttcaaagaagacacactctgttactcactgactctgttactcgcctcgac

SEQ ID NO: 33 - DNA, 117 bp, Artificial sequence
  synthetic sequence
ttacctgtggaagcaaatacttccttcaaacgctgattggctgagcaaatacttccttcaaagagtcttcaaagaagacacactctgttactcaaatactctgttactcgcctcgac

SEQ ID NO: 34 - DNA, 61 bp, Artificial sequence
  synthetic sequence
cctgtggaagcaaatacttccttcaaaaaggaagtacaaatacttccttcaaaaggtctat

SEQ ID NO: 35 - DNA, 67 bp, Artificial sequence
  synthetic sequence
cctgtggaagcaaactcagccaatcagcgcaaaaaggaagtacaaatacttccttcaaaaggtctat

SEQ ID NO: 36 - DNA, 59 bp, Artificial sequence
  synthetic sequence
cctgtggaagcaaatacttccttcaaaacttcctcaaatacttccttcaaaaggtctat

SEQ ID NO: 37 - DNA, 71 bp, Artificial sequence
  synthetic sequence
cctgtggaagcaaactcagccaatcagcgcaaaacttcctcaaactcagccaatcagcgcaaaaggtctat

SEQ ID NO: 38 - DNA, 73 bp, Artificial sequence
  synthetic sequence
cctgtggaagcaaactcagccaatcagcgcaaaaaggaagtacaaactcagccaatcagcgcaaaaggtctat

SEQ ID NO: 39 - DNA, 41 bp, Artificial sequence
  synthetic sequence
cctgtggaagcaaactcagccaatcagcgcaaaaggtctat

SEQ ID NO: 40 - DNA, 50 bp, Artificial sequence
  synthetic sequence
ccgcgactacgactctgttactcaacaactctgttactcgcctcgactgt

SEQ ID NO: 41 - DNA, 50 bp, Artificial sequence
  synthetic sequence
ccgcgactacgactctgttactcaacaactcatttactcgcctcgactgt

SEQ ID NO: 42 - DNA, 50 bp, Artificial sequence
  synthetic sequence
ccgcgactacgactctgttactcactgactctgttactcgcctcgactgt

SEQ ID NO: 43 - DNA, 50 bp, Artificial sequence
  synthetic sequence
ccgcgactacgactcatttactcactgactcatttactcgcctcgactgt

SEQ ID NO: 44 - DNA, 50 bp, Artificial sequence
  synthetic sequence
ccgcgactacgactcatttactcaacaactcatttactcgcctcgactgt

SEQ ID NO: 45 - DNA, 32 bp, Artificial sequence
  synthetic sequence
ccgcgactacgacatttactcgcctcgactgt

SEQ ID NO: 46 - DNA, 10 bp, Artificial sequence
  synthetic sequence
tgtgactcat

SEQ ID NO: 47 - DNA, 12 bp, Artificial sequence
  synthetic sequence
ggatgatgcaat

SEQ ID NO: 48 - DNA, 14 bp, Artificial sequence
  synthetic sequence
cccccgcccccgcc

SEQ ID NO: 49 - DNA, 11 bp, Artificial sequence
  synthetic sequence
atgactcagca

SEQ ID NO: 50 - DNA, 17 bp, Artificial sequence
  synthetic sequence
aaaggtcaaaggtcaac

SEQ ID NO: 51 - DNA, 13 bp, Artificial sequence
  synthetic sequence
aagatgatgtcat

SEQ ID NO: 52 - DNA, 10 bp, Artificial sequence
  synthetic sequence
gccacgccca

SEQ ID NO: 53 - DNA, 10 bp, Artificial sequence
  synthetic sequence
gaccggaagt

SEQ ID NO: 54 - DNA, 13 bp, Artificial sequence
  synthetic sequence
gaaccaggaagtg

SEQ ID NO: 55 - DNA, 7 bp, Artificial sequence
  synthetic sequence
cacgtgc

SEQ ID NO: 56 - DNA, 11 bp, Artificial sequence
  synthetic sequence
tattgcacaat

SEQ ID NO: 57 - DNA, 8 bp, Artificial sequence
  synthetic sequence
tgacgtca

SEQ ID NO: 58 - DNA, 10 bp, Artificial sequence
  synthetic sequence
ggggcggggt

SEQ ID NO: 59 - DNA, 15 bp, Artificial sequence
  synthetic sequence
ctcagccaatcagcg

SEQ ID NO: 60 - DNA, 13 bp, Artificial sequence
  synthetic sequence
aatatgcaaataa

SEQ ID NO: 61 - DNA, 15 bp, Artificial sequence
  synthetic sequence
agttaatgattaact

SEQ ID NO: 62 - DNA, 12 bp, Artificial sequence
  synthetic sequence
gttatgattaac

SEQ ID NO: 63 - DNA, 15 bp, Artificial sequence
  synthetic sequence
ctggactttggcctc

SEQ ID NO: 64 - DNA, 13 bp, Artificial sequence
  synthetic sequence
caaaggtcaagga

SEQ ID NO: 65 - DNA, 15 bp, Artificial sequence
  synthetic sequence
gtataaaaggcgggg

SEQ ID NO: 66 - DNA, 11 bp, Artificial sequence
  synthetic sequence
atgtaaacata

SEQ ID NO: 67 - DNA, 14 bp, Artificial sequence
  synthetic sequence
caaacgtaaacaat

SEQ ID NO: 68 - DNA, 15 bp, Artificial sequence
  synthetic sequence
tccatgtttactttg

SEQ ID NO: 69 - DNA, 14 bp, Artificial sequence
  synthetic sequence
ggggcccaaggggg

SEQ ID NO: 70 - DNA, 11 bp, Artificial sequence
  synthetic sequence
ccggaagtggc

SEQ ID NO: 71 - DNA, 10 bp, Artificial sequence
  synthetic sequence
accaactgtc

SEQ ID NO: 72 - DNA, 12 bp, Artificial sequence
  synthetic sequence
ggtgacgtcacc

SEQ ID NO: 73 - DNA, 14 bp, Artificial sequence
  synthetic sequence
tacgcccacgcatt

SEQ ID NO: 74 - DNA, 12 bp, Artificial sequence
  synthetic sequence
tttggcgccaaa

SEQ ID NO: 75 - DNA, 12 bp, Artificial sequence
  synthetic sequence
tgcgcacgcgca

SEQ ID NO: 76 - DNA, 193 bp, Artificial sequence
  synthetic sequence
atgacatcatcttcaaatgctgagtcatcaaacccccgcccccgcccaaatgggcgtggccaaactcagccaatcagcgcaaaaccccgcccccaaatattgcacaatcaaaaggtctatataagcagagctcgtttagtgaaccgtcagatcgcctggagacgtcgagccgagtggttgtgcctccatagaa

SEQ ID NO: 77 - DNA, 201 bp, Artificial sequence
  synthetic sequence
atgactcagcacaaatgacgtcacaaatattgcacaatcaaaatgagtcacacaaaaccccgcccccaaaattgcatcatcccaaaatgacatcatcttcaaattatttgcatattcaaaaggtctatataagcagagctcgtttagtgaaccgtcagatcgcctggagacgtcgagccgagtggttgtgcctccatagaa

SEQ ID NO: 78 - DNA, 213 bp, Artificial sequence
  synthetic sequence
caaagtaaacatggacaaaattgtttacgtttgcaaaatgtttaccaaatccttgacctttgcaaaccggaagtggccaaatacgcccacgcattcaaatacgcccacgcattcaaaccggaagtggccaaaaggtctatataagcagagctcgtttagtgaaccgtcagatcgcctggagacgtcgagccgagtggttgtgcctccatagaa

SEQ ID NO: 79 - DNA, 175 bp, Artificial sequence
  synthetic sequence
tccttgacctttgcaaaatgactcagcacaaaatgactcagcacaaatccttgacctttgcaaaatgactcagcacaaatgctgagtcatcaaaaggtctatataagcagagctcgtttagtgaaccgtcagatcgcctggagacgtcgagccgagtggttgtgcctccatagaa

SEQ ID NO: 80 - DNA, 292 bp, Artificial sequence
  synthetic sequence
gttgacctttgacctttcaaaaatatgcaaataacaaagcacgtgcaaaattgcatcatcccaaaatgagtcacacaaaatgacatcatcttcaaaattgcatcatcccaaaaggtctatataagcagagctcgtttagtgaaccgtcagatcgcctggagacgtcgagccgagtggttgtgcctccatagaagctagcctctaaggtaaatataaaatttttaagtgtataatgtgttaaactactgattctaattgtttctctcttttagattccaacctttggaactga

SEQ ID NO: 81 - DNA, 305 bp, Artificial sequence
  synthetic sequence
gttgacctttgacctttcaaaaatatgcaaataacaaagcacgtgcaaaattgcatcatcccaaaatgagtcacacaaaatgacatcatcttcaaaattgcatcatccgaacgttgacctttgacctttcaaaaatatgcaaataacaaagcacgtgcaaaattgcatcatcccaaaatgagtcacacaaaatgacatcatcttcaaaattgcatcatcccaaaaggtctatataagcagagctcgtttagtgaaccgtcagatcgcctggagacgtcgagccgagtggttgtgcctccatagaa

SEQ ID NO: 82 - DNA, 417 bp, Artificial sequence
  synthetic sequence
gttgacctttgacctttcaaaaatatgcaaataacaaagcacgtgcaaaattgcatcatcccaaaatgagtcacacaaaatgacatcatcttcaaaattgcatcatccttgggttgacctttgacctttcaaaaatatgcaaataacaaagcacgtgcaaaattgcatcatcccaaaatgagtcacacaaaatgacatcatcttcaaaattgcatcatccgaacgttgacctttgacctttcaaaaatatgcaaataacaaagcacgtgcaaaattgcatcatcccaaaatgagtcacacaaaatgacatcatcttcaaaattgcatcatcccaaaaggtctatataagcagagctcgtttagtgaaccgtcagatcgcctggagacgtcgagccgagtggttgtgcctccatagaa

SEQ ID NO: 83 - DNA, 317 bp, Artificial sequence
  synthetic sequence
tacgcccacgcattcaaaagttaatcattaactcaaatgcgcgtgcgcacaaatttggcgccaaacaaaggtgacgtcacccaaatgctgagtcatcaaacaaacgtaaacaatcaaagtataaaaggcggggcaaaaggtctatataagcagagctcgtttagtgaaccgtcagatcgcctggagacgtcgagccgagtggttgtgcctccatagaagctagcctctaaggtaaatataaaatttttaagtgtataatgtgttaaactactgattctaattgtttctctcttttagattccaacctttggaactga

SEQ ID NO: 84 - DNA, 355 bp, Artificial sequence
  synthetic sequence
tacgcccacgcattcaaaagttaatcattaactcaaatgcgcgtgcgcacaaatttggcgccaaacaaaggtgacgtcacccaaatgctgagtcatcaaacaaacgtaaacaatcaaagtataaaaggcgggggaactacgcccacgcattcaaaagttaatcattaactcaaatgcgcgtgcgcacaaatttggcgccaaacaaaggtgacgtcacccaaatgctgagtcatcaaacaaacgtaaacaatcaaagtataaaaggcggggcaaaaggtctatataagcagagctcgtttagtgaaccgtcagatcgcctggagacgtcgagccgagtggttgtgcctccatagaa

SEQ ID NO: 85 - DNA, 492 bp, Artificial sequence
  synthetic sequence
tacgcccacgcattcaaaagttaatcattaactcaaatgcgcgtgcgcacaaatttggcgccaaacaaaggtgacgtcacccaaatgctgagtcatcaaacaaacgtaaacaatcaaagtataaaaggcggggttggtacgcccacgcattcaaaagttaatcattaactcaaatgcgcgtgcgcacaaatttggcgccaaacaaaggtgacgtcacccaaatgctgagtcatcaaacaaacgtaaacaatcaaagtataaaaggcgggggaactacgcccacgcattcaaaagttaatcattaactcaaatgcgcgtgcgcacaaatttggcgccaaacaaaggtgacgtcacccaaatgctgagtcatcaaacaaacgtaaacaatcaaagtataaaaggcggggcaaaaggtctatataagcagagctcgtttagtgaaccgtcagatcgcctggagacgtcgagccgagtggttgtgcctccatagaa

SEQ ID NO: 86 - DNA, 454 bp, Artificial sequence
  synthetic sequence
tacgcccacgcattcaaaagttaatcattaactcaaatgcgcgtgcgcacaaatttggcgccaaacaaaggtgacgtcacccaaatgctgagtcatcaaacaaacgtaaacaatcaaagtataaaaggcgggggaactacgcccacgcattcaaaagttaatcattaactcaaatgcgcgtgcgcacaaatttggcgccaaacaaaggtgacgtcacccaaatgctgagtcatcaaacaaacgtaaacaatcaaagtataaaaggcggggcaaaaggtctatataagcagagctcgtttagtgaaccgtcagatcgcctggagacgtcgagccgagtggttgtgcctccatagaagctagcctctaaggtaaatataaaatttttaagtgtataatgtgttaaactactgattctaattgtttctctcttttagattccaacctttggaactga

(19) United States
(12) Patent Application Publication
(10) Pub. No.: US 2024/0209392 A1
(43) Pub. Date: Jun. 27, 2024

(54) HIGH-THROUGHPUT EXPRESSION-LINKED PROMOTER SELECTION IN EUKARYOTIC CELLS

(71) Applicant: The Regents of the University of California, Oakland, CA (US)

(72) Inventors: David V. Schaffer, Berkeley, CA (US); Joost Van Haasteren, Berkeley, CA (US); Kazuomori Lewis, Berkeley, CA (US)

(21) Appl. No.: 18/288,245

(22) PCT Filed: Apr. 25, 2022
(86) PCT No.: PCT/US22/26182

§ 371 Date: Oct. 25, 2023

Related U.S. Application Data
(60) Provisional application No. 63/179,900, filed on Apr. 26, 2021.

(51) Int. Cl. C12N 15/86; C12N 15/10

(52) U.S. Cl. CPC C12N 15/86; C12N 15/1065; C12N 15/1086; C12N 2750/14143; C12N 2830/008

(57) ABSTRACT

The present disclosure provides methods for generating synthetic transcriptional promoters that are functional in a eukaryotic cell. The present disclosure provides libraries of expression vectors comprising synthetic transcriptional promoters that are functional in a eukaryotic cell; and methods for generating the libraries. The present disclosure provides synthetic transcriptional promoters that are functional in a eukaryotic cell; as well as recombinant expression vectors comprising the synthetic transcriptional promoters.

### CROSS-REFERENCE

This application claims the benefit of U.S. Provisional Patent Application No. 63/179,900, filed Apr. 26, 2021, which application is incorporated herein by reference in its entirety.

### INCORPORATION BY REFERENCE OF SEQUENCE LISTING PROVIDED AS A TEXT FILE

A Sequence Listing is provided herewith as a text file, “BERK-448WO_SEQ_LIST_ST25.txt” created on Apr. 25, 2022, and having a size of 24 KB. The contents of the text file are incorporated by reference herein in their entirety.

### INTRODUCTION

Recombinant expression vectors find use as vehicles for delivering gene products to cells. For example, adeno-associated viruses (AAVs) have emerged as one of the most promising candidates for therapeutic DNA delivery in clinical applications. To date, AAV has been used in over 244 different clinical trials, representing 8.1% of total gene-delivery trials. Recombinant expression vectors such as AAV can be limited by packaging capacity. For example, recombinant engineered AAV has a packaging capacity of 4.7 kilobases. Various strategies to maximize the DNA packaging capacity of delivery vectors such AAV have been pursued, including attempts to increase the native packaging capacity of AAV above 4.7 kb or simply packaging more than 4.7 kb into native AAV (resulting in substantially decreased viral titers), and by reducing the length of the promoter itself.

Promoters themselves vary widely in length and strength. In general, the strongest of promoters are large; for example, the human cytomegalovirus (CMV) and the engineered CAG promoters are between 800 and 1600 base pairs in length.

There is a need in the art for synthetic promoters that are small yet retain high levels of activity.

### SUMMARY

The present disclosure provides methods for generating synthetic transcriptional promoters that are functional in a eukaryotic cell, such as a mammalian cell. The present disclosure provides libraries of expression vectors comprising synthetic transcriptional promoters that are functional in a eukaryotic cell, such as a mammalian cell; and methods for generating the libraries. The present disclosure provides synthetic transcriptional promoters that are functional in a eukaryotic cell, such as a mammalian cell; as well as recombinant expression vectors comprising the synthetic transcriptional promoters.

### BRIEF DESCRIPTION OF THE DRAWINGS

FIG. 1A-1C provide a schematic depiction of a library construction method of the present disclosure.

FIG. 2 provides a schematic depiction of barcode extraction from mRNA generated with a promoter library of the present disclosure.

FIG. 3A-3E depict construction of a promoter library. FIG. 3D depicts Cycle 1 (from top to bottom SEQ ID NOs:13, 14, 13, 13, 13, 13), Cycle 2 (from top to bottom SEQ ID NOs:15-19, 15), Cycle 3 (no Plasmidsafe; from top to bottom SEQ ID NOs:20-24, 20, 25-27, 14, 13) and Cycle 3 (with Plasmidsafe; from top to bottom SEQ ID NOs:28, 29, 28, 30, 31, 20, 23, 32, 33, 14, 14, 13). FIG. 3E depicts different promoters (from top to bottom SEQ ID NOs:34-39) and barcodes (from top to bottom SEQ ID NOs:40-45).

FIG. 4A-4C depict synthetic promoter-driven expression in HEK293T cells.

FIG. 5 depicts differences in percent identity of TFBS motifs in plasmid vs. extracted mRNA.

FIG. 6 depicts green fluorescent protein (GFP) expression from individual clones in the 3× TFBS experiment.

FIG. 7 depicts transfection analysis of synthetic promoters generated from ubiquitous promoter libraries.

FIG. 8 depicts transduction analysis of synthetic promoters generated from ubiquitous promoter libraries.

FIG. 9 presents Table 1, which provides TFBS motifs present in Ubiquitous Library 1 (from top to bottom SEQ ID NOs:46-60) and Ubiquitous Library 2 (from top to bottom SEQ ID NOs:61-71, 49, 72-75).

FIG. 10 presents Table 2, which provides nucleotide sequences of examples of synthetic promoters of the present disclosure (from top to bottom SEQ ID NOs:76, 11, 77, 78, 12, 79).

FIG. 11 depicts the architecture of modular ELiPS promoters.

FIG. 12A-12B present charts showing that modular ELiPS promoter activity is improved in plasmid transfection.

FIG. 13 presents Table 3, which provides sequences of modular ELiPS promoter variants.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding sequence if the promoter affects its transcription or expression.

A “vector” or “expression vector” is a replicon, such as plasmid, phage, virus, or cosmid, to which another DNA segment, i.e. an “insert”, may be attached so as to bring about the replication and/or expression of the attached segment in a cell.

“Heterologous,” as used herein, means a nucleotide or polypeptide sequence that is not found in the native (e.g., naturally-occurring) nucleic acid or protein, respectively.

The term “genetic modification” refers to a permanent or transient genetic change induced in a cell following introduction into the cell of a heterologous nucleic acid (e.g., a nucleic acid exogenous to the cell). Genetic change (“modification”) can be accomplished by incorporation of the heterologous nucleic acid into the genome of the host cell, or by transient or stable maintenance of the heterologous nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, and the like.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a transcription factor binding site” includes a plurality of such transcription factor binding sites and reference to “the core promoter” includes reference to one or more core promoters and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods for generating synthetic transcriptional promoters that are functional in a eukaryotic cell, such as a mammalian cell. The present disclosure provides libraries of expression vectors comprising synthetic transcriptional promoters that are functional in a eukaryotic cell, such as a mammalian cell; and methods for generating the libraries. The present disclosure provides synthetic transcriptional promoters that are functional in a eukaryotic cell, such as a mammalian cell; as well as recombinant expression vectors comprising the synthetic transcriptional promoters.

### METHODS OF GENERATING A SYNTHETIC TRANSCRIPTIONAL PROMOTER

The present disclosure provides methods for generating synthetic transcriptional promoters that are functional in a eukaryotic cell, such as a mammalian cell.

The methods comprise: A) introducing an expression vector into a eukaryotic cell, such as a mammalian cell, where the expression vector comprises: a) a synthetic transcriptional promoter comprising: i) a first transcription factor binding site (TFBS) comprising an upstream enhancer element of from 4 to 20 base pairs (bp) in length; ii) at least a second TFBS, where the at least a second TFBS comprises an upstream enhancer element of from 4 to 20 bp and has a nucleotide sequence that is the same or different from the first TFBS; and iii) a core promoter comprising: a TATA box; an initiator element; an RNA Polymerase II binding site; and a transcription start site; and b) a nucleotide sequence encoding a reporter polypeptide, wherein the nucleotide sequence encoding the reporter polypeptide is operably linked to the synthetic transcriptional promoter; and B) detecting expression of the reporter polypeptide. Expression of the reporter polypeptide in the eukaryotic cell (e.g., the mammalian cell) indicates that the synthetic transcriptional promoter that is functional in the eukaryotic cell (e.g., the mammalian cell). In some cases, the at least a second TFBS has a nucleotide sequence that is different from the first TFBS. In some cases, the at least a second TFBS has a nucleotide sequence that is the same as that of the first TFBS. Additional TFBS can be inserted into the vector, where each subsequent TFBS is inserted immediately 3′ of the previously-inserted TFBS, generating an expression vector comprising a synthetic transcriptional promoter comprising: i) multiple TFBS (e.g., multiple tandem TFBS); and ii) a core promoter. In some cases, an expression vector generated by the method comprises from 2 to 30 TFBSs.

### Barcodes

In some cases, the expression vector comprises a nucleic acid barcode that identifies the combination of the from 2 to 30 TFBS. The nucleic acid barcode is 3′ of the nucleotide sequence encoding the reporter polypeptide. The nucleic acid barcode is a composite of barcodes that identify the individual TFBS. Thus, e.g., where the expression vector comprises a first TFBS, a second TFBS, and a third TFBS, the composite barcode will comprise a first barcode (BC) that identifies the first TFBS, a second BC that identifies the second TFBS, and a third BC that identifies the third TFBS.

### TFBS

In some cases, the expression vector comprises from 2 to 30 TFBSs. For example, in some cases, the expression vector comprises from 2 to 5 TFBS, from 2 to 10 TFBSs, from 5 to 10 TFBSs, from 10 to 15 TFBSs, from 15 to 20 TFBSs, or from 20 to 30 TFBSs. For example, in some cases, the expression vector comprises: i) a first TFBS; ii) a second TFBS; and iii) a third TFBS, where the first, second, and third TFBS differ from one another in nucleotide sequence. As another example, in some cases, the expression vector comprises: i) a first TFBS; ii) a second TFBS; and iii) a third TFBS, where the 2 or more of the first, second, and third TFBS have the same nucleotide sequence. As another example, in some cases, the expression vector comprises: i) a first TFBS; ii) a second TFBS; iii) a third TFBS; and iv) a fourth TFBS, where the first, second, third, and fourth TFBS differ from one another in nucleotide sequence. As another example, in some cases, the expression vector comprises: i) a first TFBS; ii) a second TFBS; iii) a third TFBS; and iv) a fourth TFBS, where 2 or more of the first, second, third, and fourth TFBS have the same nucleotide sequence. As another example, in some cases, the expression vector comprises: i) a first TFBS; ii) a second TFBS; iii) a third TFBS; iv) a fourth TFBS; and v) a fifth TFBS, where the first, second, third, fourth, and fifth TFBS differ from one another in nucleotide sequence. As another example, in some cases, the expression vector comprises: i) a first TFBS; ii) a second TFBS; iii) a third TFBS; iv) a fourth TFBS; and v) a fifth TFBS, where 2 or more of the first, second, third, fourth, and fifth TFBS have the same nucleotide sequence (e.g., 2 of the TFBSs have the same nucleotide sequence; and the other 5 differ from one another in nucleotide sequence, and differ in nucleotide sequence from the 2 that share the same nucleotide sequence). The TFBS functions as an upstream enhancer.

Each of the TFBS independently has a length of from about 4 bp to about 20 bp. For example, each of the TFBS independently has a length of 4 bp, 5 bp, 6 bp, 7 bp, 8 bp, 9 bp, 10 bp, 11 bp, 12 bp, 13 bp, 14 bp, 15 bp, 16 bp, 17 bp, 18 bp, 19 bp, or 20 bp.

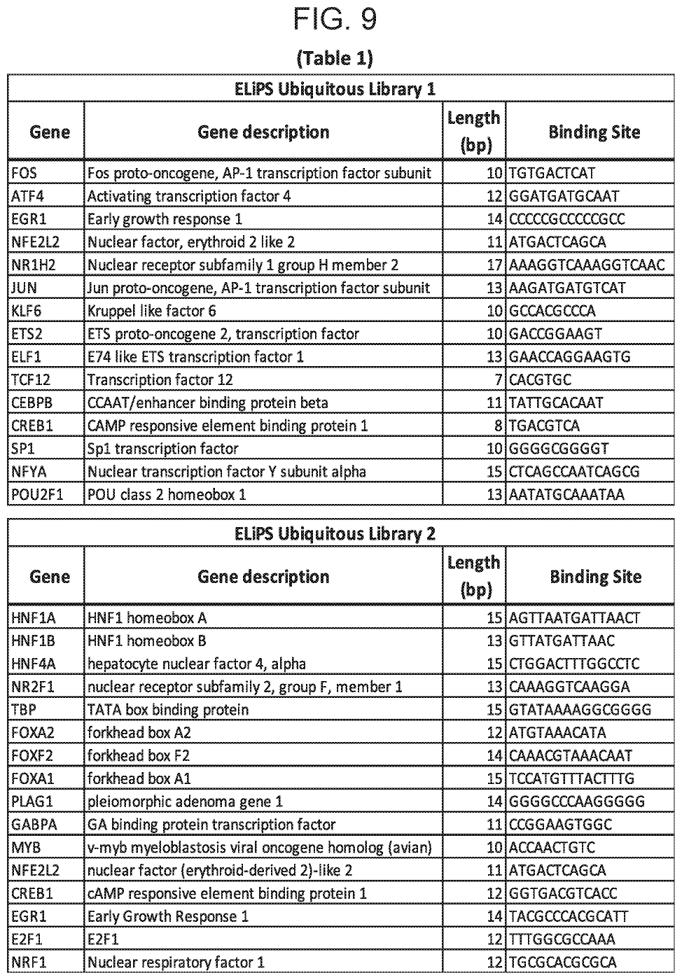

TFBSs can be selected from any of various public databases. Non-limiting examples of suitable TFBSs are depicted in Table 1 (FIG. 9). Examples of TFBSs include binding sites for transcription factors such as, e.g., JUN, NFE2L2, EGR1, KLF6, NFYA, SP1, CEBPB, NR1H2, POU2F, TCF12, ATF4, FOS, CREB1, FOXA1, FOXF2, FOXD1, NR2F1, GABPA, HNF1A, NRF1, E2F1, FBP, and the like.

TFBS can be or any origin, e.g., from any eukaryotic cell, e.g., a plant cell, an insect cell, a mammalian cell, an arthropod cell, an amphibian cell, a reptile cell, a fish cell, an avian cell, and the like. In some cases, the TFBSs are mammalian cell origin. In some cases, the TFBSs comprise one or more nucleotide sequence differences from a naturally-occurring TFBS.

### Core Promoter

The core promoter comprises: i) a TATA box; ii) an initiator element; iii) an RNA Polymerase II binding site; and iv) a transcription start site. Suitable core promoters are known in the art; and any core promoter can be used. The core promoter can have a length of from about 50 nucleotides (nt) to about 150 nt. For example, the core promoter can have a length of from about 50 nt to about 60 nt, from about 60 nt to about 70 nt, from about 70 nt to about 80 nt, from about 80 nt to about 90 nt, from about 90 nt to about 100 nt, from about 100 nt to about 110 nt, from about 110 nt to about 120 nt, from about 120 nt to about 130 nt, or from about 130 nt to about 150 nt.

As one non-limiting example, an SCP2 core promoter can be used. For example, an SCP2 core promoter can have the following nucleotide sequence:

(SEQ ID NO: 1)
AGGTCTATATAAGCAGAGCTCGTTTAGTGAACCGTCAGATCGCCTGGAG
ACGTCGAGCCGAGTGGTTGTGCCTCCATAGAA;

and can have a length of 81 nucleotides (nt).

As another non-limiting example, an SCP1 core promoter can be used. For example, an SCP1 core promoter can have the following nucleotide sequence:

(SEQ ID NO: 2)
GTACTTATATAAGGGGGTGGGGGCGCGTTCGTCCTCAGTCGCGATCGAA
CACTCGAGCCGAGCAGACGTGCCTACGGACCG;

and can have a length of 81 nucleotides.

As another non-limiting example, a cytomegalovirus (CMV) IE1 core promoter can be used. For example, a CMV IE1 core promoter can have the following nucleotide sequence:

(SEQ ID NO: 3)
AGGTCTATATAAGCAGAGCTCGTTTAGTGAACCGTCAGATCGCCTGGAG
ACGCCATCCACGCTGTTTTGACCTCCATAGAA;

and can have a length of 81 nucleotides.

As another non-limiting example, a core promoter can have the following nucleotide sequence:

(SEQ ID NO: 4)
AGGAGGTGGGGGACCCAGAGGGGCTTTGACGTCAGCCTGGCCTTTAAGA
GGCCGCCTGCCTGGCAAGGGCTGTGGAGACAGAACTCGGGACCACCAGC
TT;

and can have a length of 100 nucleotides.

In some cases, the core promoter is a ubiquitous promoter; i.e., the promoter is functional in a wide variety of cell types. In some cases, the core promoter is a cell type-specific promoter; i.e., the promoter is functional in one type of cell, or a limited number of cell types. For example, a core promoter can be a hepatocyte-specific promoter, a cardiac cell-specific promoter, a glial cell-specific promoter, a neuron-specific promoter, a skeletal muscle cell-specific promoter, a T cell-specific promoter, a B cell-specific promoter, or the like.

The synthetic transcriptional promoter (including the two or more TFBS and the core promoter) generally has a length of from about 90 nucleotides (nt) to about 800 nt. For example, the synthetic transcriptional promoter generally has a length of from about 90 nt to about 100 nt, from about 100 nt to about 150 nt, from about 150 nt to about 175 nt, from about 175 nt to about 200 nt, from about 175 nt to about 225 nt, from about 190 nt to about 220 nt, from about 200 nt to about 250 nt, from about 250 nt to about 300 nt, from about 300 nt to about 350 nt, from about 350 nt to about 400 nt, from about 400 nt to about 450 nt, from about 450 nt to about 500 nt, from about 500 nt to about 550 nt, from about 550 nt to about 600 nt, from about 600 nt to about 650 nt, from about 650 nt to about 700 nt, from about 700 nt to about 750 nt, from about 750 nt to about 800 nt, from about 800 nt to about 850 nt, or from about 850 nt to 900 nt.

### Reporter Polypeptides

Suitable reporter polypeptides include, e.g., a fluorescent polypeptide; an enzyme that acts on a substrate to produce a fluorescent product, a luminescent product, or a colored product; a cell surface polypeptide; a functional polypeptide; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

As noted above, in some cases, the reporter polypeptide is a polypeptide that is expressed on the cell surface. Detection of such a reporter polypeptide can be carried out using an antibody (e.g., a detectably labeled antibody) specific for the reporter polypeptide.

Also suitable for use as a reporter polypeptide are polypeptides that provide for a function in a eukaryotic cell. In some cases, the function is selectable (e.g., drug resistance).

### Libraries of Expression Vectors Comprising Synthetic Transcriptional Promoters

The present disclosure provides libraries of expression vectors comprising synthetic transcriptional promoters that are functional in a eukaryotic cell (e.g., a mammalian cell).

A library of expression vectors comprises a plurality of expression vector members, each member expression vector comprising: a) a synthetic transcriptional promoter comprising: i) a first TFBS comprising an upstream enhancer element of from 4 to 20 base pairs (bp) in length; ii) at least a second TFBS, wherein the at least a second TFBS comprises an upstream enhancer element of from 4 to 20 bp and has a nucleotide sequence that is the same or different from the first TFBS; and iii) a core promoter comprising: a TATA box; an initiator element; an RNA Polymerase II binding site; and a transcription start site; and b) a nucleotide sequence encoding a reporter polypeptide, wherein the nucleotide sequence encoding the reporter polypeptide is operably linked to the synthetic transcriptional promoter.

In some cases, each member expression vector independently comprises from 2 to 30 TFBSs. For example, in some cases, a member expression vector comprises from 2 to 5 TFBS, from 2 to 10 TFBSs, from 5 to 10 TFBSs, from 10 to 15 TFBSs, from 15 to 20 TFBSs, or from 20 to 30 TFBSs.

The synthetic transcriptional promoter (including the two or more TFBS and the core promoter) generally has a length of from about 90 nucleotides (nt) to about 800 nt. For example, the synthetic transcriptional promoter generally has a length of from about 90 nt to about 100 nt, from about 100 nt to about 150 nt, from about 150 nt to about 175 nt, from about 175 nt to about 200 nt, from about 175 nt to about 225 nt, from about 190 nt to about 220 nt, from about 200 nt to about 250 nt, from about 250 nt to about 300 nt, from about 300 nt to about 350 nt, from about 350 nt to about 400 nt, from about 400 nt to about 450 nt, from about 450 nt to about 500 nt, from about 500 nt to about 550 nt, from about 550 nt to about 600 nt, from about 600 nt to about 650 nt, from about 650 nt to about 700 nt, from about 700 nt to about 750 nt, from about 750 nt to about 800 nt, from about 800 nt to about 850 nt, or from about 850 nt to 900 nt.

Suitable reporter polypeptides are as described above. Suitable reporter polypeptides include, e.g., a fluorescent polypeptide; an enzyme that acts on a substrate to produce a fluorescent product, a luminescent product, or a colored product; a cell surface polypeptide; a functional polypeptide; and the like.

A subject library can have from 10² to 10¹¹ or more different member recombinant expression vectors. For example, a subject library can have from about 10² to about 10⁴, from about 10⁴ to about 10⁶, from about 10⁶ to about 10⁷, from about 10⁷ to about 10⁸, from about 10⁸ to about 10⁹, from about 10⁹ to about 10¹⁰, or from about 10¹⁰ to about 10¹¹, or more than 10¹¹ different member recombinant expression vectors.

### Methods for Generating a Library of Expression Vectors Comprising Synthetic Transcriptional Promoters

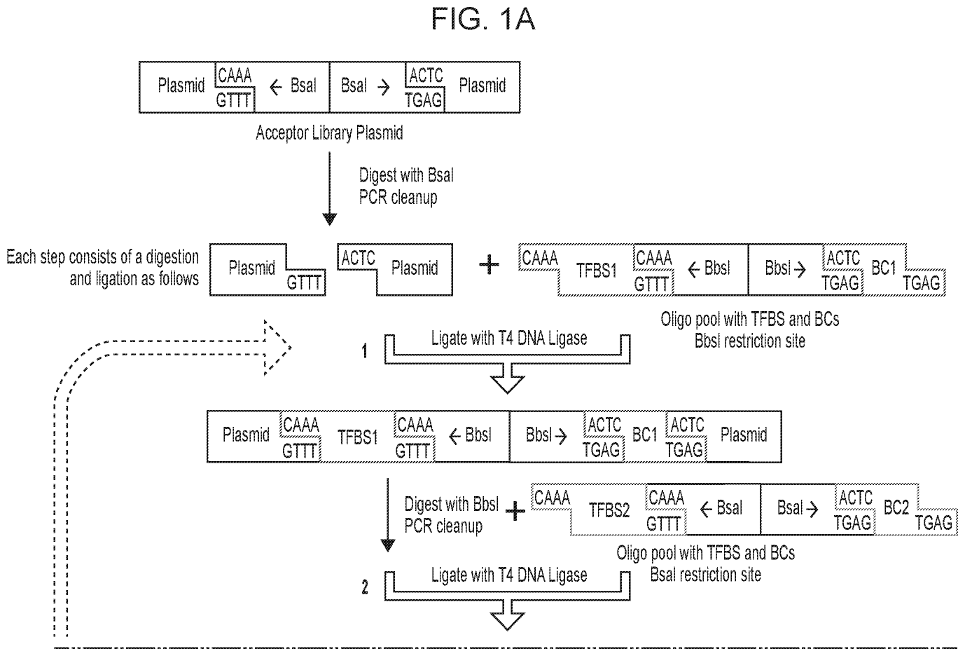

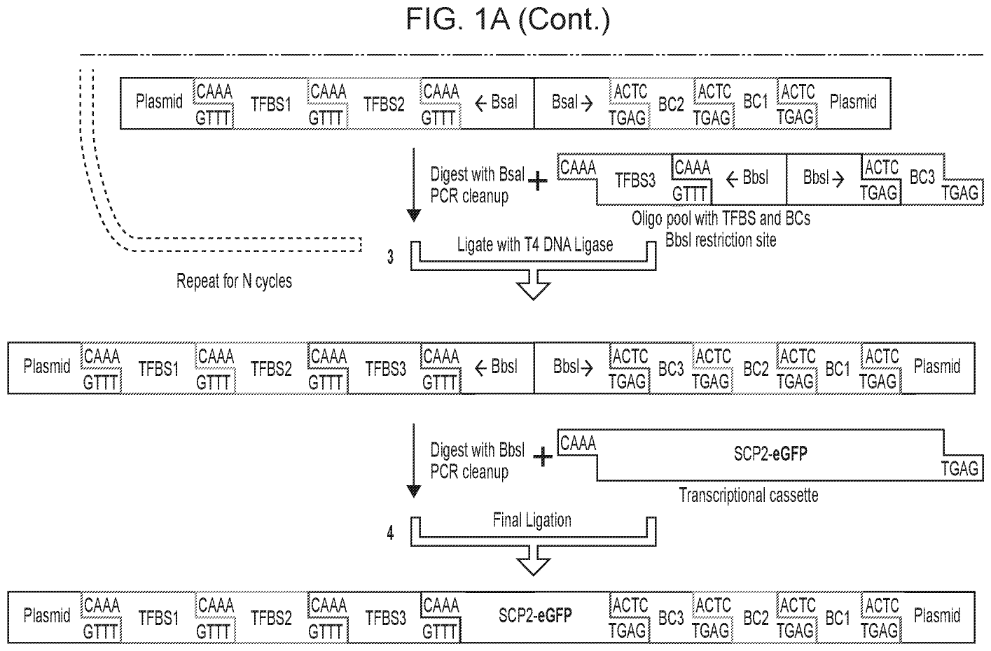

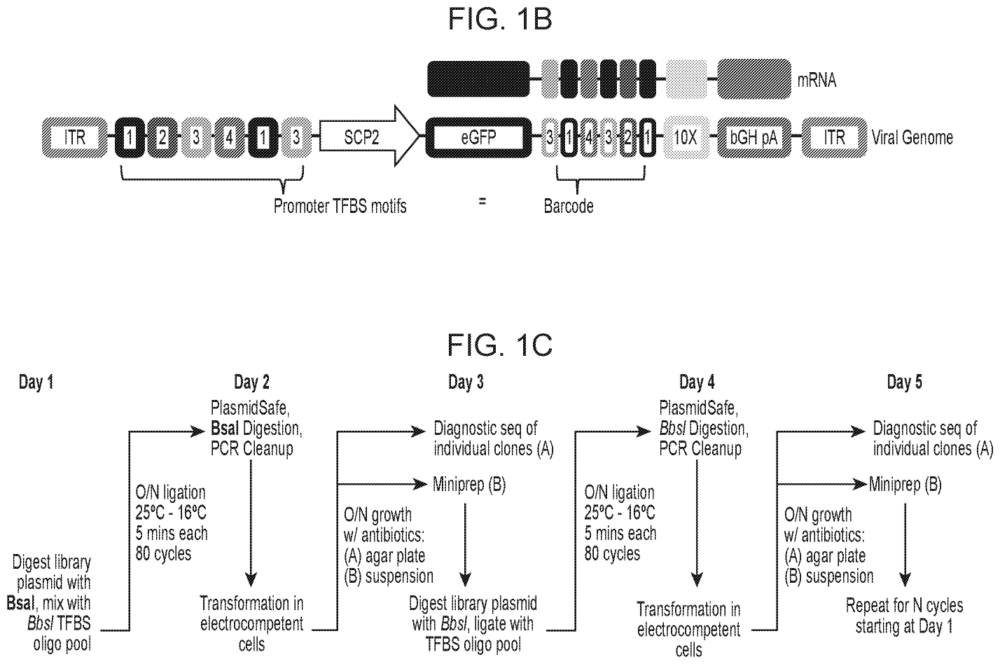

The present disclosure provides methods for generating a library of expression vectors comprising synthetic transcriptional promoters that are functional in a eukaryotic cell (e.g., a mammalian cell). The methods comprise: a) introducing into an expression vector a first nucleic acid comprising: i) a first TFBS comprising an upstream enhancer element of from 4 to 20 base pairs (bp) in length; ii) a first restriction enzyme recognition site; and iii) a first barcode that identifies the first TFBS, wherein the first restriction enzyme site is not present elsewhere in the expression vector, and wherein said introducing results in a first modified expression vector; b) cleaving the first modified expression vector with a restriction enzyme that cleaves the first restriction enzyme recognition site, generating a first linear modified expression vector; c) ligating to the first linear modified expression vector a second nucleic acid comprising: i) a second TFBS comprising an upstream enhancer element of from 4 to 20 bp in length; ii) a second restriction enzyme recognition site; and iii) a second barcode, wherein: the second TFBS has the same nucleotide sequence or a different in nucleotide sequence from the first TFBS, the second restriction enzyme site is not present elsewhere in the expression vector and is different from the first restriction enzyme site, and the second barcode identifies the second TFBS; wherein said ligating results in a second modified expression vector; d) cleaving the second modified expression vector with a restriction enzyme that cleaves the second restriction enzyme recognition site, resulting in a second linear modified expression vector; and e) ligating to second linear modified expression vector a nucleic acid comprising: i) a core promoter; and ii) a nucleotide sequence encoding a reporter polypeptide, wherein said ligating results in a recombinant expression vector comprising: i) a synthetic transcriptional promoter comprising at least two TFBSs and the core promoter; and ii) a composite barcode comprising the two barcodes, wherein the composite barcode identifies the two TFBSs, and wherein the composite barcode is 3′ of the nucleotide sequence encoding the reporter polypeptide. The general method is depicted schematically in FIG. 1A-1C. Example 1 provides an example as to how the method can be carried out.

In some cases, the restriction enzymes that are used are selected such that, following digestion with that restriction enzyme, the original restriction enzyme recognition site is removed. For example, in some cases, Type IIS restriction enzymes are used. As one non-limiting example, the first restriction enzyme recognition site is cleaved by BbsI and the second restriction enzyme recognition site is cleaved by BsaI.

The nucleic acid comprising the TFBS and the restriction enzyme recognition site can be from a pool of nucleic acids that differ from one another in the TFBS, but that have the same restriction enzyme recognition site. The pool can have from about 2 to about 10⁶ different TFBS in combination with the same restriction enzyme recognition site. For example, the pool can have from about 2 to about 10, from about 10 to about 15, from about 15 to about 20, from about 20 to about 25, from about 25 to about 50, from about 50 to about 10², from about 10² to about 10⁴, or from about 10⁴ to about 10⁶, different TFBS in combination with the same restriction enzyme recognition site. The pool can have from about 10² to about 10⁴, or from about 10⁴ to about 10⁶, different TFBS in combination with the same restriction enzyme recognition site. Thus, the same TFBS can theoretically be inserted in subsequent ligation steps, or different TFBS can be inserted in subsequent ligation steps.

For example, the method can comprise repeating steps (a) through (c) to insert at least a third nucleic acid comprising: i) a third TFBS comprising an upstream enhancer element of from 4 to 20 base pairs (bp) in length; ii) the first restriction enzyme recognition site; and iii) a third barcode, thereby generating a recombinant expression vector comprising: i) a synthetic transcriptional promoter comprising at least three TFBSs and the core promoter; and ii) a composite barcode comprising the three barcodes, wherein the composite barcode identifies the three TFBSs. In addition, the method can comprise repeating steps (a) through (c) to generate a recombinant expression vector comprising: i) a synthetic transcriptional promoter comprising from 4 to 10 TFBSs and the core promoter; and ii) a composite barcode that identifies the collection of from 4 to 10 TFBSs.

TFBSs can be selected from any of various public databases. Non-limiting examples of suitable TFBSs are depicted in Table 1 (FIG. 9). Examples of TFBSs include binding sites for transcription factors such as, e.g., JUN, NFE2L2, EGR1, KLF6, NFYA, SP1, CEBPB, NR1H2, POU2F, TCF12, ATF4, FOS, CREB1, FOXA1, FOXF2, FOXD1, NR2F1, GABPA, HNF1A, NRF1, E2F1, FBP, and the like. In some cases, the TFBSs inserted at each step that involves insertion of a nucleic acid comprising a TFBS are independently selected from TFBSs depicted in FIG. 9.

The synthetic transcriptional promoter (including the two or more TFBS and the core promoter) generally has a length of from about 90 nucleotides (nt) to about 800 nt. For example, the synthetic transcriptional promoter generally has a length of from about 90 nt to about 100 nt, from about 100 nt to about 150 nt, from about 150 nt to about 175 nt, from about 175 nt to about 200 nt, from about 175 nt to about 225 nt, from about 190 nt to about 220 nt, from about 200 nt to about 250 nt, from about 250 nt to about 300 nt, from about 300 nt to about 350 nt, from about 350 nt to about 400 nt, from about 400 nt to about 450 nt, from about 450 nt to about 500 nt, from about 500 nt to about 550 nt, from about 550 nt to about 600 nt, from about 600 nt to about 650 nt, from about 650 nt to about 700 nt, from about 700 nt to about 750 nt, from about 750 nt to about 800 nt, from about 800 nt to about 850 nt, or from about 850 nt to 900 nt.

Suitable reporter polypeptides include, e.g., a fluorescent polypeptide; an enzyme that acts on a substrate to produce a fluorescent product, a luminescent product, or a colored product; a cell surface polypeptide; a functional polypeptide; and the like, as described above. In some cases, the reporter polypeptide is a fluorescent protein. In some cases, the reporter polypeptide is an enzyme that produces a fluorescent product, a luminescent product, or a colored product. In some cases, the reporter polypeptide is a cell surface polypeptide.

The present disclosure provides a method of producing a library of recombinant expression vectors, each comprising a different synthetic transcriptional promoter, the method comprising carrying out the method as described above with a plurality of expression vectors, to generate a library of recombinant expression vectors, each comprising a different synthetic transcriptional promoter, each with a unique composite barcode that appears 3′ of the nucleotide sequence encoding the reporter polypeptide. In some cases, the method comprises introducing members of the library into eukaryotic host cells (e.g., mammalian host cells), and determining whether the reporter polypeptide is expressed in one or more of the eukaryotic host cells (e.g., mammalian host cells).

The barcode is cloned into the vector in such a way that it is present on the 3′ end of the untranslated region (UTR) of each mRNA molecule. The strength of the promoter is directly proportional to the number of transcripts it produces, which is also proportional to the number of times a particular barcode is recovered from the RNA. In some cases, a cDNA copy of the mRNA transcripts generated by transcription driven by the synthetic transcriptional promoter is made. In some cases, generation of the cDNA copy introduces into the cDNA a unique molecular identifier (UMI), and in some cases polymerase chain reaction (PCR) amplification sequence. Such a process allows one to tag individual mRNA molecules with an UMI such that it can be demultiplexed after PCR amplification, preparing samples for next generation sequencing (NGS). In that way, individual mRNA molecules can be counted, and individual barcodes tied directly to expression from their corresponding promoter.

### Synthetic Transcriptional Promoters

The present disclosure provides synthetic transcriptional promoters that are functional in a eukaryotic cell (e.g., a mammalian cell).

In some cases, a functional synthetic transcriptional promoter of the present disclosure comprises: i) a nucleotide sequence having at least 90%, at least 95%, at least 98%, at least 99%, or 100%, nucleotide sequence identity to the following nucleotide sequence:

(SEQ ID NO: 5)
ATGACATCATCTTCAAATGCTGAGTCATCAAACCCCCGCCCCCGCCCAA
ATGGGCGTGGCCAAACTCAGCCAATCAGCGCAAAACCCCGCCCCCAAAT
ATTGCACAAT;

and ii) a core promoter.

In some cases, a functional synthetic transcriptional promoter of the present disclosure comprises: i) a nucleotide sequence having at least 90%, at least 95%, at least 98%, at least 99%, or 100% nucleotide sequence identity to the following nucleotide sequence:

(SEQ ID NO: 6)
GTTGACCTTTGACCTTTCAAAAATATGCAAATAACAAAGCACGTGCAAA
ATTGCATCATCCCAAAATGAGTCACACAAAATGACATCATCTTCAAAAT
TGCATCATCC;

and ii) a core promoter.

In some cases, a functional synthetic transcriptional promoter of the present disclosure comprises: i) a nucleotide sequence having at least 90%, at least 95%, at least 98%, at least 99%, or

100%, nucleotide sequence identity to the following nucleotide sequence:

(SEQ ID NO: 7)
ATGACTCAGCACAAATGACGTCACAAATATTGCACAATCAAAATGAGTC
ACACAAAACCCCGCCCCCAAAATTGCATCATCCCAAAATGACATCATCT
TCAAATTATTTGCATATT;

and ii) a core promoter.

In some cases, a functional synthetic transcriptional promoter of the present disclosure comprises: i) a nucleotide sequence having at least 90%, at least 95%, at least 98%, at least 99%, or 100%, nucleotide sequence identity to the following nucleotide sequence:

(SEQ ID NO: 8)
CAAAGTAAACATGGACAAAATTGTTTACGTTTGCAAAATGTTTACCAAA
TCCTTGACCTTTGCAAACCGGAAGTGGCCAAATACGCCCACGCATTCAA
ATACGCCCACGCATTCAAACCGGAAGTGGC;

and ii) a core promoter.

In some cases, a functional synthetic transcriptional promoter of the present disclosure comprises: i) a nucleotide sequence having at least 90%, at least 95%, at least 98%, at least 99%, or 100%, nucleotide sequence identity to the following nucleotide sequence:

(SEQ ID NO: 9)
TACGCCCACGCATTCAAAAGTTAATCATTAACTCAAATGCGCGTGCGCA
CAAATTTGGCGCCAAACAAAGGTGACGTCACCCAAATGCTGAGTCATCA
AACAAACGTAAACAATCAAAGTATAAAAGGCGGGG;

and ii) a core promoter.

In some cases, a functional synthetic transcriptional promoter of the present disclosure comprises: i) a nucleotide sequence having at least 90%, at least 95%, at least 98%, at least 99%, or 100%, nucleotide sequence identity to the following nucleotide sequence:

(SEQ ID NO: 10)
TCCTTGACCTTTGCAAAATGACTCAGCACAAAATGACTCAGCACAAATC
CTTGACCTTTGCAAAATGACTCAGCACAAATGCTGAGTCAT;

and ii) a core promoter.

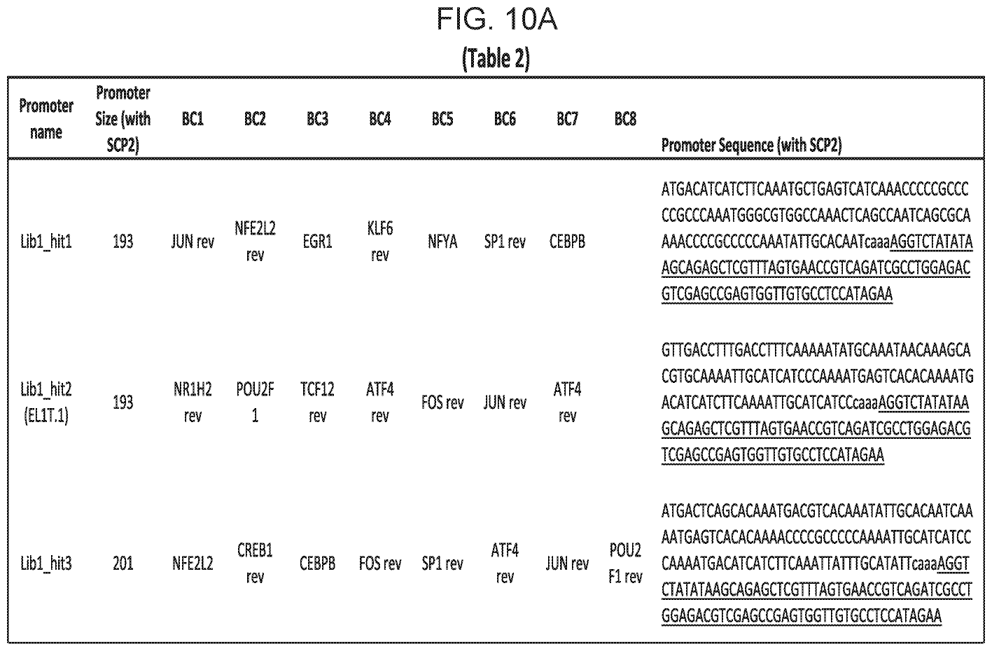

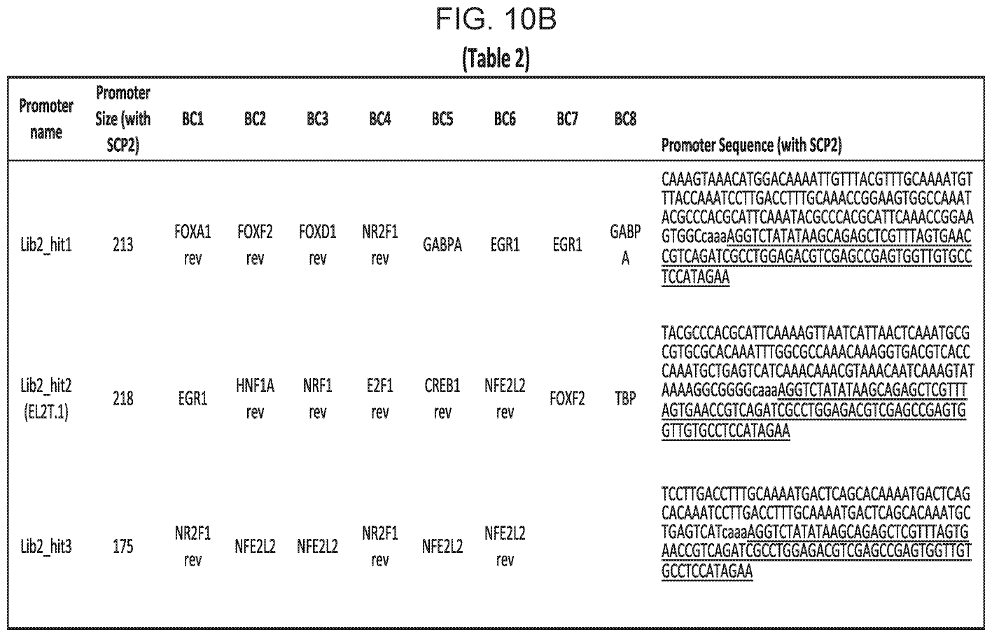

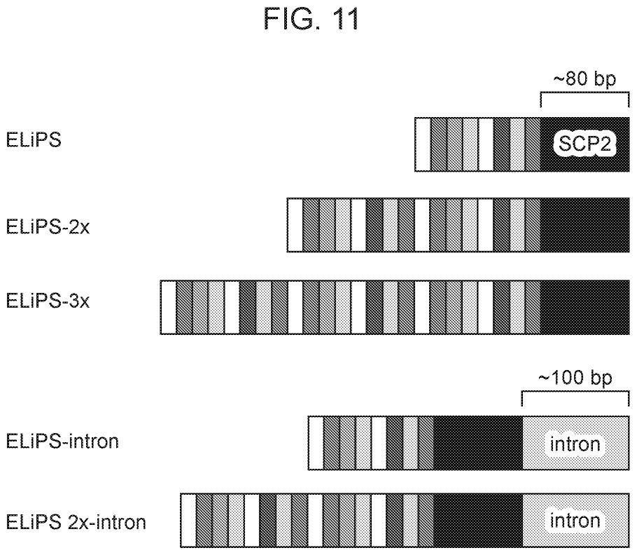

In some cases, a functional synthetic transcriptional promoter of the present disclosure comprises a nucleotide sequence having at least 90% nucleotide sequence identity to any one of the nucleotide sequences depicted in FIG. 10 (Table 2). In some cases, a functional synthetic transcriptional promoter of the present disclosure comprises a nucleotide sequence having at least 90%, at least 95%, at least 98%, at least 99%, or 100%, nucleotide sequence identity to any one of the nucleotide sequences depicted in FIG. 10 (Table 2).

In some cases, a functional synthetic transcriptional promoter of the present disclosure comprises a nucleotide sequence having at least 90%, at least 95%, at least 98%, at least 99%, or 100%, nucleotide sequence identity to the nucleotide sequence identified as “EL1T.1” in Table 2 (FIG. 10). In some cases, a functional synthetic transcriptional promoter of the present disclosure comprises the nucleotide sequence:

(SEQ ID NO: 11)
GTTGACCTTTGACCTTTCAAAAATATGCAAATAACAAAGCACGTGCAAA
ATTGCATCATCCCAAAATGAGTCACACAAAATGACATCATCTTCAAAAT
TGCATCATCCcaaaAGGTCTATATAAGCAGAGCTCGTTTAGTGAACCGT
CAGATCGCCTGGAGACGTCGAGCCGAGTGGTTGTGCCTCCATAGAA,

where the core promoter is underlined.

In some cases, a functional synthetic transcriptional promoter of the present disclosure comprises a nucleotide sequence having at least 90%, at least 95%, at least 98%, at least 99%, or 100%, nucleotide sequence identity to the nucleotide sequence identified as “EL2T.1” in Table 2 (FIG. 10). In some cases, a functional synthetic transcriptional promoter of the present disclosure comprises the nucleotide sequence:

(SEQ ID NO: 12)
TACGCCCACGCATTCAAAAGTTAATCATTAACTCAAATGCGCGTGCGCA
CAAATTTGGCGCCAAACAAAGGTGACGTCACCCAAATGCTGAGTCATCA
AACAAACGTAAACAATCAAAGTATAAAAGGCGGGGcaaaAGGTCTATAT
AAGCAGAGCTCGTTTAGTGAACCGTCAGATCGCCTGGAGACGTCGAGCC
GAGTGGTTGTGCCTCCATAGAA,

where the core promoter is underlined.

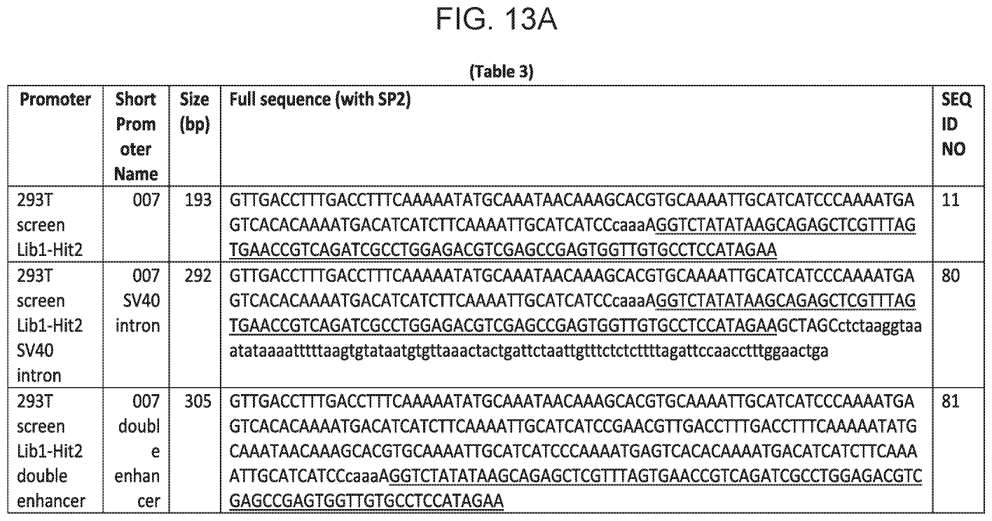

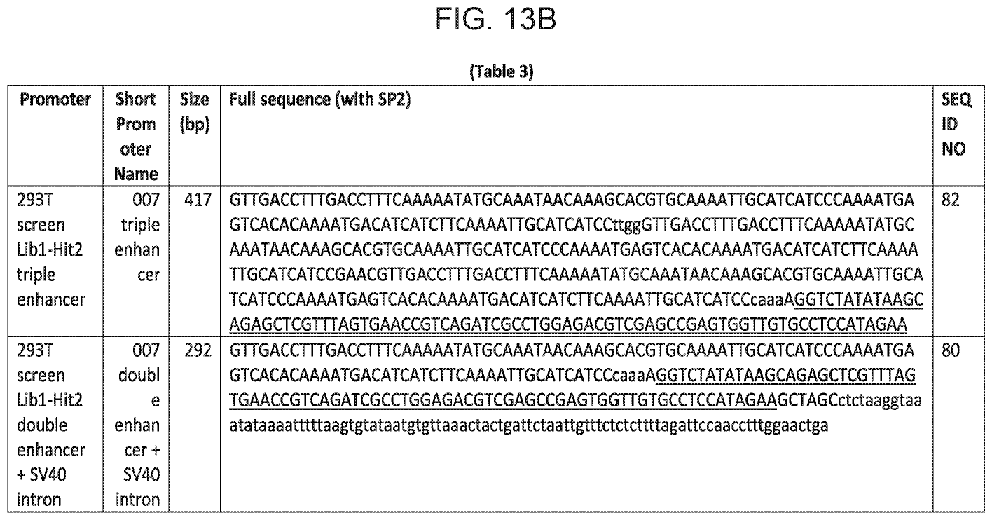

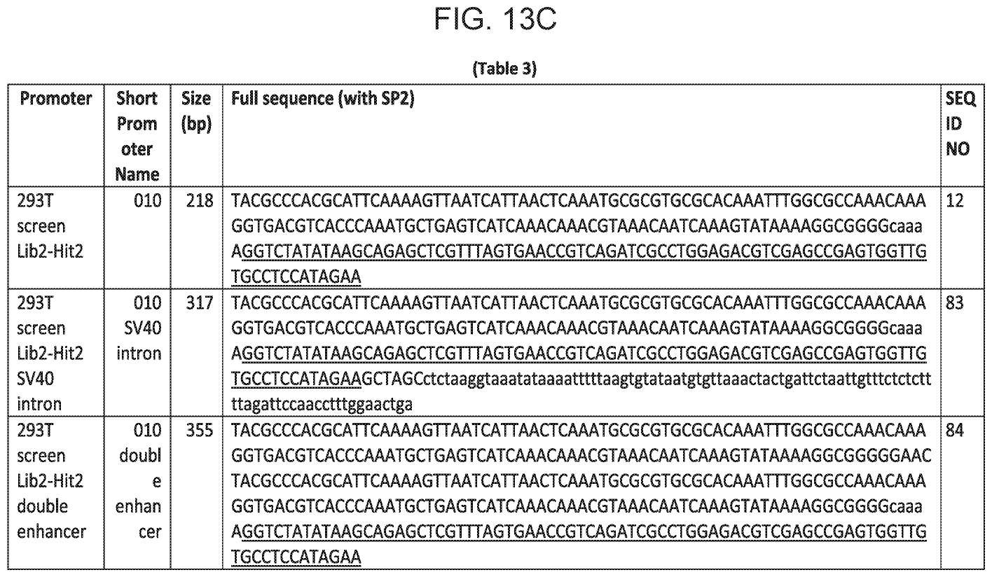

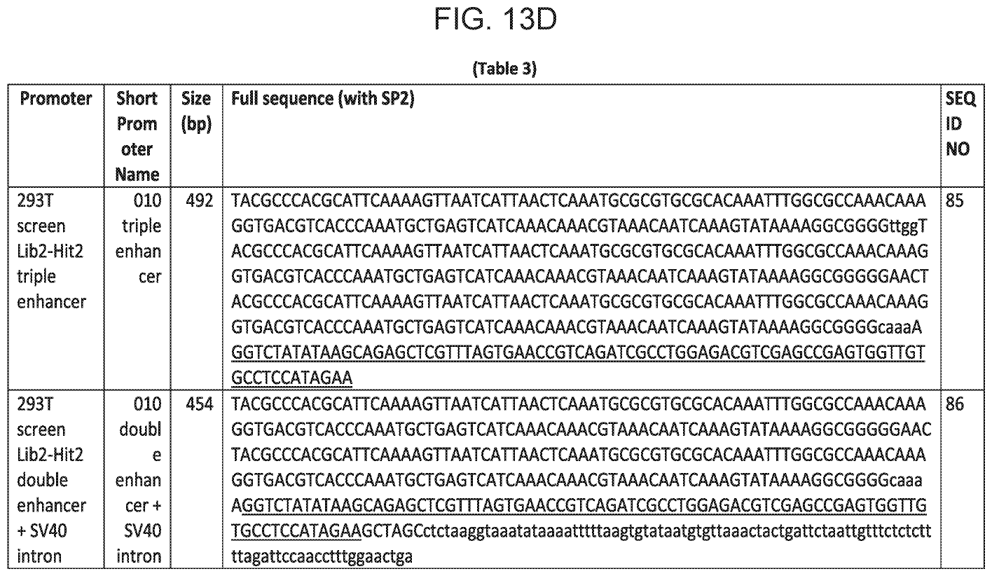

In some cases, a functional synthetic transcriptional promoter of the present disclosure comprises a nucleotide sequence having at least 90% nucleotide sequence identity to any one of the nucleotide sequences depicted in FIG. 13 (Table 3). In some cases, a functional synthetic transcriptional promoter of the present disclosure comprises a nucleotide sequence having at least 90%, at least 95%, at least 98%, at least 99%, or 100%, nucleotide sequence identity to any one of the nucleotide sequences depicted in FIG. 13 (Table 3; SEQ ID NO:11, SEQ ID NO:12, and SEQ ID NOs:80-86). In some cases, a functional synthetic transcriptional promoter of the present disclosure comprises a nucleotide sequence having at least 90%, at least 95%, at least 98%, at least 99%, or 100%, nucleotide sequence identity to the nucleotide sequence set forth in SEQ ID NO:80. In some cases, a functional synthetic transcriptional promoter of the present disclosure comprises a nucleotide sequence having at least 90%, at least 95%, at least 98%, at least 99%, or 100%, nucleotide sequence identity to the nucleotide sequence set forth in SEQ ID NO:81. In some cases, a functional synthetic transcriptional promoter of the present disclosure comprises a nucleotide sequence having at least 90%, at least 95%, at least 98%, at least 99%, or 100%, nucleotide sequence identity to the nucleotide sequence set forth in SEQ ID NO:82. In some cases, a functional synthetic transcriptional promoter of the present disclosure comprises a nucleotide sequence having at least 90%, at least 95%, at least 98%, at least 99%, or 100%, nucleotide sequence identity to the nucleotide sequence set forth in SEQ ID NO:83. In some cases, a functional synthetic transcriptional promoter of the present disclosure comprises a nucleotide sequence having at least 90%, at least 95%, at least 98%, at least 99%, or 100%, nucleotide sequence identity to the nucleotide sequence set forth in SEQ ID NO:84. In some cases, a functional synthetic transcriptional promoter of the present disclosure comprises a nucleotide sequence having at least 90%, at least 95%, at least 98%, at least 99%, or 100%, nucleotide sequence identity to the nucleotide sequence set forth in SEQ ID NO:85. In some cases, a functional synthetic transcriptional promoter of the present disclosure comprises a nucleotide sequence having at least 90%, at least 95%, at least 98%, at least 99%, or 100%, nucleotide sequence identity to the nucleotide sequence set forth in SEQ ID NO:86.

### Recombinant Expression Vectors

The present disclosure provides recombinant expression vectors comprising a synthetic transcriptional promoter of the present disclosure. A recombinant expression vector of the present disclosure comprises a vector into which a synthetic transcriptional promoter of the present disclosure has been inserted.

In some cases, a recombinant expression vector of the present disclosure comprises an insertion site (e.g., a restriction enzyme recognition site) 3′ of the synthetic transcriptional promoter (e.g., within about 100 nucleotides (nt), within about 50 nt, within about 25 nt, or within about 10 nt) 3′ of the synthetic transcriptional promoter), for insertion of a nucleic acid comprising a nucleotide sequence encoding a gene product(s) of interest. Gene products include polypeptides, RNAs, and combinations thereof. For example, a nucleic acid comprising a nucleotide sequence encoding a gene product of interest comprises a nucleotide sequence encoding a CRISPR/Cas effector polypeptide and a corresponding guide RNA.

In some cases, a recombinant expression vector of the present disclosure comprises: i) a synthetic transcriptional promoter of the present disclosure; and ii) a nucleic acid comprising a nucleotide sequence encoding a gene product(s) of interest, where the nucleic acid is operably linked to the synthetic transcriptional promoter.

Vectors which may be used include, without limitation, lentiviral, retroviral, herpes simplex virus (HSV), adenoviral, and adeno-associated viral (AAV) vectors. Lentivirus vectors include, but are not limited to vectors based on human immunodeficiency virus (e.g., HIV-1, HIV-2), simian immunodeficiency virus (SIV), feline immunodeficiency virus (FIV), and equine infectious anemia virus (EIAV). Lentiviruses may be pseudotyped with the envelope proteins of other viruses, including, but not limited to vesicular stomatitis virus (VSV), rabies virus, Moloney-murine leukemia virus (Mo-MLV), baculovirus, and Ebola virus. Such vectors may be prepared using standard methods in the art. Retroviruses include, but are not limited to Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus, and the like.

In some cases, a suitable vector is a recombinant AAV vector. AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome that contains the rep gene involved in viral replication and expression of the viral genes; and the right-hand part of the genome that contains the cap gene encoding the capsid proteins of the virus.

In some cases, the recombinant vector is encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the present disclosure includes a recombinant virus particle (recombinant because it contains a recombinant polynucleotide) comprising any of the vectors described herein. Methods of producing such particles are known in the art and are described in U.S. Pat. No. 6,596,535, the disclosure of which is hereby incorporated by reference in its entirety.

### Compositions

A recombinant expression vector of the present disclosure can be present in a nanoparticle, a micelle, a vesicle, or a liposome. Thus, the present disclosure comprises a composition comprising: i) a recombinant expression vector of the present disclosure; and ii) a nanoparticle, a micelle, a vesicle, or a liposome.

A recombinant expression vector of the present disclosure can be present in a composition with one or more of a lipid, a polysaccharide, and a polymer. Thus, the present disclosure comprises a composition comprising: i) a recombinant expression vector of the present disclosure; and ii) one or more of: a cationic lipid, a neutral lipid, an anionic lipid, a polysaccharide, and a polymer. Suitable cationic lipids include, e.g., N,N-dioleyl-N,N-dimethylammonium chloride (DODAC), N,N-distearyl-N,N-dimethylammonium bromide (DDAB), N-(1-(2,3-dioleoyloxy) propyl)-N,N,N-trimethylammonium chloride (DOTAP), 1,2-Dioleoyl-3-Dimethylammonium-propane (DODAP), N-(1-(2,3-dioleyloxy)propyl)-N,N,N-trimethylammonium chloride (DOTMA), 1,2-Dioleoylcarbamyl-3-Dimethylammonium-propane (DOCDAP), 1,2-Dilineoyl-3-Dimethylammonium-propane (DLINDAP), dilauryl(C₁₂:0) trimethyl ammonium propane (DLTAP), Dioctadecylamidoglycyl spermine (DOGS), DC-Choi, Dioleoyloxy-N-[2-sperminecarboxamido)ethyl}-N,N-dimethyl-1-propanaminiumt-rifluoroacetate (DOSPA), 1,2-Dimyristyloxypropyl-3-dimethyl-hydroxyethyl ammonium bromide (DMRIE), 3-Dimethylamino-2-(Cholest-5-en-3-beta-oxybutan-4-oxy)-1-(cis,cis-9,12-oc-tadecadienoxy)propane (CLinDMA), N,N-dimethyl-2,3-dioleyloxy)propylamine (DODMA), 2-[5′-(cholest-5-en-3[beta]-oxy)-3′-oxapentoxy)-3-dimethyl-1-(ci-s,cis-9′,12′-octadecadienoxy) propane (CpLinDMA) and N,N-Dimethyl-3,4-dioleyloxybenzylamine (DMOBA), and 1,2-N,N′-Dioleylcarbamyl-3-dimethylaminopropane (DOcarbDAP).

Suitable neutral lipids include, e.g., 5-heptadecylbenzene-1,3-diol (resorcinol), dipalmitoylphosphatidylcholine (DPPC), distearoylphosphatidylcholine (DSPC), phosphocholine (DOPC), dimyristoylphosphatidylcholine (DMPC), phosphatidylcholine (PLPC), I,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), I-myristoyl-2-palmitoyl phosphatidylcholine (MPPC), I-palmitoyl-2-myristoyl phosphatidylcholine (PMPC), I-palmitoyl-2-stearoyl phosphatidylcholine (PSPC), I,2-diarachidoyl-sn-glycero-3-phosphocholine (DBPC), I-stearoyl-2-palmitoyl phosphatidylcholine (SPPC), I,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine, distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid is selected from the group consisting of distearoylphosphatidylcholine (DSPC) and dimyristoyl phosphatidyl ethanolamine (DMPE).

Anionic lipids suitable for inclusion in a composition of the present disclosure include, but are not limited to, phosphatidylglycerol, cardiolipin, diacylphosphatidylserine, diacylphosphatidic acid, N-dodecanoyl phosphatidyl ethanoloamine, N-succinyl phosphatidylethanolamine, N-glutaryl phosphatidylethanolamine cholesterol hemisuccinate (CHEMS), and lysylphosphatidylglycerol.

In some cases, a composition of the present disclosure comprises one or more polymers. Suitable polymers include polyamines, dendrimers, and copolymers. Suitable polymers include, e.g., polyethylene glycol, polyglycolide, polyvinyl alcohol, polyvinyl pyrrolidone, polylactide, poly(lactide-co-glycolide), polycaprolactone, polysorbate, polyethylene oxide, polypropylene oxide, poly(ethylene oxide-co-propylene oxide), poloxamer, poloxamine, poly(oxyethylated) glycerol, poly(oxyethylated) sorbitol, poly(oxyethylated) glucose, and polyethyleneimine. Suitable polymers include polysaccharides. In some cases, the polymer is polyethyleneimine (PEI). In some cases, the polymer is polyamidoamine (PAMAM) dendrimer. In some cases, the polymer is poly(lactide-co-glycolide) (PLGA). In some cases, the polymer is the block copolymer poly(ethylene glycol)-block-poly(lactic-co-glycolic acid) (PEG-b-PLGA).

### Genetically Modified Host Cells

The present disclosure provides genetically modified host cells, e.g., genetically modified eukaryotic cells comprising a synthetic transcriptional promoter of the present disclosure. The present disclosure provides genetically modified host cells, e.g., genetically modified eukaryotic cells comprising a recombinant expression vector of the present disclosure.

Cells that can be genetically modified cell with a synthetic transcriptional promoter of the present disclosure or with a recombinant expression vector of the present disclosure include: single-cell eukaryotic organisms; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell from a vertebrate animal (e.g., a fish, an amphibian, a reptile, a bird, a mammal); a cell from a mammal (e.g., a cell from a rodent; a cell from a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuna, a sheep, a goat, etc.); a cell of a marine mammal (e.g., a whale, a seal, an elephant seal, a dolphin, a sea lion; etc.) and the like. Any type of cell may be of interest (e.g. a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell, a retinal cell, a lung epithelial cell; an in vitro or in vivo embryonic cell of an embryo at any stage, e.g., a 1-cell, 2-cell, 4-cell, 8-cell, etc. stage zebrafish embryo; etc.). In some cases, the cell is a cell that does not originate from a natural organism (e.g., the cell can be a synthetically made cell; also referred to as an artificial cell). In some cases, the cell is a mammalian cell (e.g., a human cell, a non-human primate cell, etc.).

In some cases, the cell is part of a multicellular organism (e.g., a plant, an animal, etc.). In some cases, the cell is in an organoid.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1 A method for generating a synthetic transcriptional promoter that is functional in a eukaryotic cell, the method comprising: A) introducing an expression vector into a eukaryotic cell, wherein the expression vector comprises: a) a synthetic transcriptional promoter comprising: i) a first transcription factor binding site (TFBS) comprising an upstream enhancer element of from 4 to 20 base pairs (bp) in length; ii) at least a second TFBS, wherein the at least a second TFBS comprises an upstream enhancer element of from 4 to 20 bp and has a nucleotide sequence that is the same or different from the first TFBS; and iii) a core promoter comprising: a TATA box; an initiator element; an RNA Polymerase II binding site; and a transcription start site; and b) a nucleotide sequence encoding a reporter polypeptide, wherein the nucleotide sequence encoding the reporter polypeptide is operably linked to the synthetic transcriptional promoter; and B) detecting expression of the reporter polypeptide, wherein expression of the reporter polypeptide in the eukaryotic cell indicates that the synthetic transcriptional promoter that is functional in the eukaryotic cell.

Aspect 2. The method of aspect 1, wherein the expression vector comprises from 2 to 30 TFBS.

Aspect 3. The method of aspect 2, wherein the expression vector comprises a nucleic acid barcode that identifies the combination of the from 2 to 30 TFBS.

Aspect 4. The method of any one of aspects 1-3, wherein the synthetic transcriptional promoter has a length of no more than about 700 bp.

Aspect 5. The method of aspect 4, wherein the synthetic transcriptional promoter has a length of from 100 bp to about 700 bp.

Aspect 6. The method of any one of aspects 1-5, wherein the reporter polypeptide is a fluorescent protein.

Aspect 7. The method of any one of aspects 1-5, wherein the reporter polypeptide is an enzyme that produces a fluorescent product, a luminescent product, or a colored product.

Aspect 8. The method of any one of aspects 1-5, wherein the reporter polypeptide is a cell surface polypeptide.

Aspect 9. The method of any one of aspects 1-8, comprising determining the nucleotide sequence of the functional synthetic transcriptional promoter.

Aspect 10. The method of any one of aspects 1-9, wherein the core promoter is a ubiquitous promoter.

Aspect 11. The method of any one of aspects 1-9, wherein the core promoter is a cell type-specific promoter.

Aspect 12. A library of expression vectors comprising a plurality of members comprising: a) a synthetic transcriptional promoter comprising: i) a first transcription factor binding site (TFBS) comprising an upstream enhancer element of from 4 to 20 base pairs (bp) in length; ii) at least a second TFBS, wherein the at least a second TFBS comprises an upstream enhancer element of from 4 to 20 bp and has a nucleotide sequence that is the same or different from the first TFBS; and iii) a core promoter comprising: a TATA box; an initiator element; an RNA Polymerase II binding site; and a transcription start site; and b) a nucleotide sequence encoding a reporter polypeptide, wherein the nucleotide sequence encoding the reporter polypeptide is operably linked to the synthetic transcriptional promoter.

Aspect 13. The library of aspect 12, wherein the expression vector comprises from 2 to 30 TFBS.

Aspect 14. The library of aspect 13, wherein the expression vector comprises a nucleic acid barcode that identifies the combination of the from 2 to 30 TFBS.

Aspect 15. The library of any one of aspects 12-14, wherein the synthetic transcriptional promoter has a length of no more than about 700 bp.

Aspect 16. The library of aspect 15, wherein the synthetic transcriptional promoter has a length of from 100 bp to about 700 bp.

Aspect 17. The library of any one of aspects 12-16, wherein the reporter polypeptide is a fluorescent protein.

Aspect 18. The library of any one of aspects 12-16, wherein the reporter polypeptide is an enzyme that produces a fluorescent product, a luminescent product, or a colored product.

Aspect 19. The library of any one of aspects 12-16, wherein the reporter polypeptide is a cell surface polypeptide.

Aspect 20. The library of any one of aspects 12-19, wherein the library comprises from 10² to 10¹¹ members.

Aspect 21. A functional synthetic transcriptional promoter comprising a nucleotide sequence having at least 90%, at least 95%, at least 98%, at least 99%, or 100%, nucleotide sequence identity to any one of the nucleotide sequences depicted in FIG. 10 or FIG. 13.

Aspect 22. The functional synthetic transcriptional promoter of aspect 21, comprising a nucleotide sequence having at least 95%, at least 98%, at least 99%, or 100%, nucleotide sequence identity to the promoter sequence identified as ELIT.1 in FIG. 10.

Aspect 23. The functional synthetic transcriptional promoter of aspect 21, comprising a nucleotide sequence having at least 95%, at least 98%, at least 99%, or 100%, nucleotide sequence identity to the promoter sequence identified as EL2T.1 in FIG. 10.

Aspect 24. A recombinant expression vector comprising the synthetic transcriptional promoter of any one of aspects 21-23.

Aspect 25. The recombinant expression vector of aspect 24, wherein the synthetic transcriptional promoter is operably linked to a nucleotide sequence encoding a polypeptide of interest.

Aspect 26. The recombinant expression vector of aspect 24 or aspect 25, wherein the vector is an adeno-associated virus (AAV) vector.

Aspect 27. The recombinant expression vector of aspect 24 or aspect 25, wherein the vector is a lentivirus vector or an adenovirus vector.

Aspect 28. A composition comprising the recombinant expression vector of any one of aspects 24-27.

Aspect 29. The composition of aspect 28, comprising a nanoparticle, a lipid, or a liposome.

Aspect 30. A eukaryotic cell genetically modified with:
a) the functional synthetic transcriptional promoter of any one of aspects 21-23;
b) the recombinant expression vector of any one of aspects 24-27.

Aspect 31. The eukaryotic cell of aspect 30, wherein the cell is a mammalian cell.

Aspect 32. A method of generating a recombinant expression vector comprising a synthetic transcriptional promoter, the method comprising: a) introducing into an expression vector a first nucleic acid comprising: i) a first transcription factor binding site (TFBS) comprising an upstream enhancer element of from 4 to 20 base pairs (bp) in length; ii) a first restriction enzyme recognition site; and iii) a first barcode that identifies the first TFBS, wherein the first restriction enzyme site is not present elsewhere in the expression vector, wherein said introducing results in a first modified expression vector; b) cleaving the first modified expression vector with a restriction enzyme that cleaves the first restriction enzyme recognition site, generating a first linear modified expression vector; c) ligating to the first linear modified expression vector a second nucleic acid comprising: i) a second TFBS comprising an upstream enhancer element of from 4 to 20 base pairs (bp) in length; ii) a second restriction enzyme recognition site; and iii) a second barcode, wherein: the second TFBS has the same nucleotide sequence or a different in nucleotide sequence from the first TFBS, the second restriction enzyme site is not present elsewhere in the expression vector and is different from the first restriction enzyme site, and the second barcode identifies the second TFBS; wherein said ligating results in a second modified expression vector; d) cleaving the second modified expression vector with a restriction enzyme that cleaves the second restriction enzyme recognition site, resulting in a second linear modified expression vector; and e) ligating to second linear modified expression vector a nucleic acid comprising: i) a core promoter; and ii) a nucleotide sequence encoding a reporter polypeptide, wherein said ligating results in a recombinant expression vector comprising: i) a synthetic transcriptional promoter comprising at least two TFBSs and the core promoter; and ii) a composite barcode comprising the two barcodes, wherein the composite barcode identifies the two TFBSs, wherein the composite barcode is 3′ of the nucleotide sequence encoding the reporter polypeptide.

Aspect 33. The method of aspect 32, further comprising repeating steps (a) through (c) to insert at least a third nucleic acid comprising: i) a third TFBS comprising an upstream enhancer element of from 4 to 20 base pairs (bp) in length; ii) the first restriction enzyme recognition site; and iii) a third barcode, thereby generating a recombinant expression vector comprising: i) a synthetic transcriptional promoter comprising at least three TFBSs and the core promoter; and ii) a composite barcode comprising the three barcodes, wherein the composite barcode identifies the three TFBSs.

Aspect 34. The method of aspect 32, further comprising repeating steps (a) through (c) to generate a recombinant expression vector comprising: i) a synthetic transcriptional promoter comprising from 4 to 30 TFBSs and the core promoter; and ii) a composite barcode.

Aspect 35. The method of any one of aspects 32-34, wherein the first restriction enzyme recognition site is cleaved by BbsI and wherein the second restriction enzyme recognition site is cleaved by BsaI.

Aspect 36. The method of any one of aspects 32-35, wherein the TFBSs are independently selected from TFBSs depicted in FIG. 9.

Aspect 37. The method of any one of aspects 32-36, wherein the synthetic transcriptional promoter has a length of no more than about 700 bp.

Aspect 38. The method of aspect 37, wherein the synthetic transcriptional promoter has a length of from 100 bp to about 700 bp.

Aspect 39. The method of any one of aspects 32-38, wherein the reporter polypeptide is a fluorescent protein.

Aspect 40. The method of any one of aspects 32-38, wherein the reporter polypeptide is an enzyme that produces a fluorescent product, a luminescent product, or a colored product.

Aspect 41. The method of any one of aspects 32-38, wherein the reporter polypeptide is a cell surface polypeptide.

Aspect 42. A method of producing a library of recombinant expression vectors, each comprising a different synthetic transcriptional promoter, the method comprising carrying out the method of any one of aspects 32-41 with a plurality of expression vectors, to generate a library of recombinant expression vectors, each comprising a different synthetic transcriptional promoter, each with a unique composite barcode.

Aspect 43. The method of aspect 42, further comprising introducing members of the library into eukaryotic host cells, and determining whether the reporter polypeptide is expressed in one or more of the eukaryotic host cells.

Aspect 44. The method of aspect 43, comprising determining the nucleotide sequence of the composite barcode.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Generation and Characterization of Synthetic Transcriptional Promoters

The following example describes a platform for the efficient generation of large (>10⁷) libraries of synthetic promoters that can be functionally screened using AAV vectors for the high throughput selection of promoters based on their expression properties in cells or tissues of interest. Through this method (termed “ELiPS” (Expression-Linked Promoter Selection)), synthetic promoters are built sequentially from small transcription factor binding site (TFBS) motifs in coordinated steps, allowing precise control of promoter size. ELiPS enables the construction of synthetic promoter libraries in which a barcode in the 3′ UTR of the mRNA transcript is directly linked to the identity of the promoter that drove its expression, which allows for signal amplification of desirable promoters. Its design is amenable to next generation sequencing analysis of promoter strength. The general strategy is depicted in FIG. 1A-1C.

FIG. 1A-1C. The ELiPS method of the construction of a promoter library consisting of tandem copies of TFBS binding motifs creates a direct linkage between the TFBS motifs present in the promoter and barcode sequences in the 3′ UTR region of the mRNA transcribed by that promoter (A). To do that, pools of oligos containing a TFBS and unique 4 bp barcode sequence are ligated into an acceptor plasmid in multiple cycles, where the number of cycles determines how many TFBS motifs are present in the promoter. By integrating type IIS restriction sites in the oligos, each subsequent oligo will be seamlessly inserted between the TFBS motif and BC sequence of the previous cycle's ligation product. Two pools of oligos are created that contain the same TFBS/BC combinations but distinct restriction sites (BsaI and BbsI). Starting with BsaI (1), each subsequent cycle flips between BbsI and BsaI to increase the number of TFBS motifs (2 and 3). This creates a library of N TFBS motifs in tandem (1-2-N) followed by N barcodes in reverse orientation (N-2-1). After the last cycle, a transcription cassette is ligated into the library (4). mRNA molecules driven by transcription from a certain promoter will have the exact identity reflected in the 3′ UTR of the mRNA molecule itself (B). A schematic of the protocol's day by day process is shown in (C).

TFBS motifs can be selected using any desired method or databases (Ex: CHIP-seq, ATAC-seq, experimental or published data, etc.). For the purposes of this initial test of ubiquitous promoters, selected TFBS motifs were selected using a combination of the FANTOMS & JASPAR (ELiPS library 2), and the Human Protein Atlas (ELiPS library 1) databases as follows. TFBS were selected using a combination of the FANTOMS database (https://fantom.gsc.riken.jp/5/sstar/Main_Page) and the Human Protein Atlas. In the FANTOMS database, mRNA datasets comprising tissue and cell types of interest were analyzed through Cap Analysis of Gene Expression (CAGE) to select TFBS motifs (10-14 base pair sequences) that were over-represented in the proximal region of promoters that were active in total RNA pool samples (TFBS motifs selected were p<0.0001). Subsequently, a literature search was performed to remove hits whose associated TFs were implicated in any sort of repressive or inflammatory activity, as well as those requiring protein complexes of larger than 4 transcription factor subunits to drive downstream gene expression. Lastly, the updated versions of each of the selected TFBS motifs were derived from the JASPAR database (http://jaspar.genereg.net/, version 2020). For the initial ubiquitous library, three different ‘human reference’ mRNA datasets were used. TFBS motif selections for ELiPS library 2 can be found in Table 1 (FIG. 9).

Table 2 (FIG. 10). Top promoters from ubiquitous ELiPS libraries. TFBS identity and location of each motif comprising the top six ubiquitous promoters. BC denotes barcode location in the promoter, and a “_rev” indication denotes the binding site for that particular TF was in reverse (3′-5′) orientation. Between each TBFS motif, there is an ‘ACTC’ sequence used as a spacer. In each promoter, the SCP2 sequence is underlined.

From the Protein Atlas database (https://followed by: www(dot)proteinatlas(dot)org)), expression values of genes annotated as “Transcription Factors” were downloaded from all available tissues. To find TFs with high and ubiquitous expression, the average of the normalized expression value per gene was calculated for all tissues (60 tissue types in total). To select against TFs expressed at very high levels in just a small number of tissues, a situation that would skew the average, the median and geometric mean were also calculated and only transcription factors with >5 normalized expression values in all three columns were selected for further analysis (Table 1; FIG. 9). A literature search on the resulting transcription factors was performed, and genes that were implicated in immune responses and/or could have negative transcriptional activities through post-translational modification were removed from the final TF pool. Updated TFBS sequences were derived from the JASPAR database or through a literature search. TFBS motif selections for ELiPS library 1 can be found in Table 1 (FIG. 9).

The ELiPS method of the construction of a promoter library consisting of tandem copies of TFBS binding motifs creates a direct linkage between the TFBS motifs present in the promoter and barcode sequences in the 3′ untranslated region (3′ UTR) of the mRNA transcribed by that promoter (FIG. 1A-1C). To do that, pools of oligonucleotides (“oligos”) containing a TFBS and unique 4 bp barcode sequence were ligated into an acceptor plasmid in multiple cycles, where the number of cycles determines how many TFBS motifs are present in the promoter. By integrating type IIS restriction sites in the oligos, each subsequent oligo was ligated between the TFBS motif and barcode sequence of the previous cycle's ligation product. Two pools of oligos were created that contain the same TFBS/BC combinations but distinct restriction sites (BsaI and BbsI). Starting with BsaI (step 1), each subsequent cycle flips between BbsI and BsaI to increase the number of TFBS motifs (steps 2 and 3). This created a library of N TFBS motifs in tandem (1-2-N) followed by N barcodes in reverse orientation (N-2-1). After the last cycle, a transcription cassette was ligated into the library (step 4). mRNA molecules driven by transcription from a certain promoter will have the exact identity reflected in the 3′ UTR of the mRNA molecule itself (FIG. 1B). Subsequently, promoters that drive strong levels of expression will produce larger numbers of mRNA molecules containing the promoter's barcode ID. A schematic of the day-by-day cloning protocol is shown in (FIG. 1C).

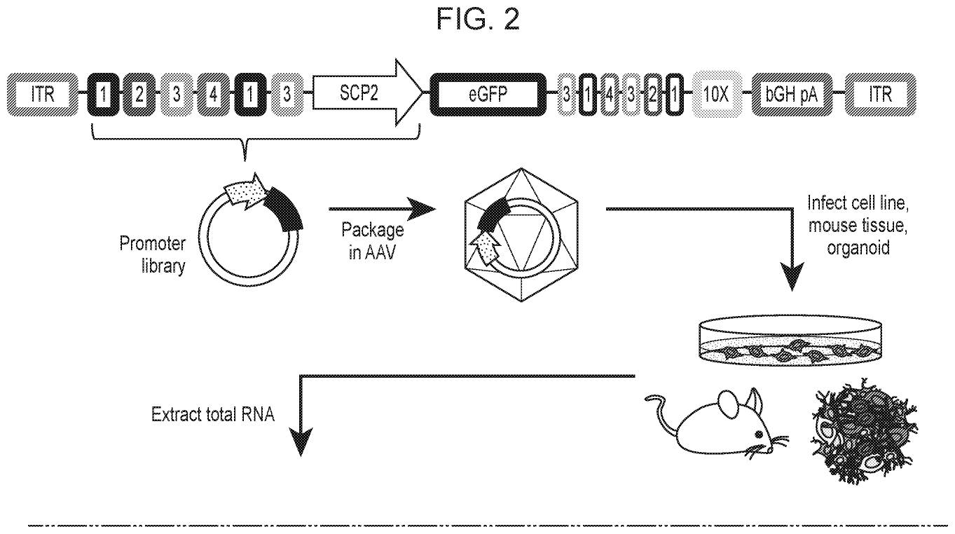

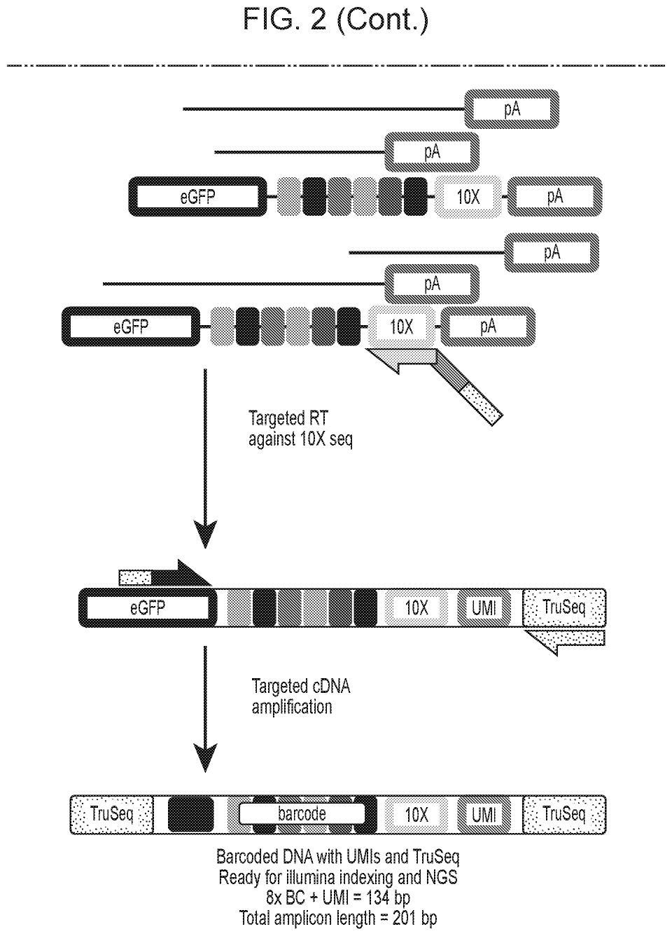

To identify promising promoter candidates, the sequence of the barcode array in the 3′ UTR the transcribed mRNA was determined. Total RNA was extracted after an appropriate time duration depending on the delivery method and vehicle (e.g. 72 hours for transfection in cell culture and 1-2 weeks for in vivo transduction with AAV). This total RNA was then converted to cDNA using a reverse transcription (RT) primer that is specific to the promoter library mRNA, resulting in targeted reverse transcription (RT) of the mRNA of interest only (FIG. 2). The cDNA was then amplified. The RT primer contained a unique molecular identifier (UMI) to reduce polymerase chain reaction (PCR) bias that could otherwise impact accurate counting of individual mRNA molecules. The resulting amplicon containing the barcode (BC) sequences relating to promoter identity and unique molecular identifier (UMI) was then sequenced on an Illumina platform and fed into a bioinformatics pipeline. This pipeline extracts the barcode sequences from the individual reads and then removes the duplicate reads caused by the PCR amplification based on both the UMI and BC identities. The resulting data represents the barcode content in the cell from which the mRNA is extracted and is fed into further analysis tools to identify highly prevalent TFBS motifs and overrepresented combinations.

FIG. 2. Targeted barcode extraction from ELiPS mRNA. Cells or tissues are transfected or transduced with a plasmid or virus containing a ELiPS promoter library. After an appropriate amount of time dependent on the vector and model, total RNA was extracted. This total RNA was then converted to cDNA using an RT primer that is specific to the promoter library mRNA—In this case, this unique sequence is the 10× capture sequence, making this process also amenable to use with single cell RNA sequencing. The result is targeted reverse transcription (RT) of the mRNA of interest only. The cDNA is then amplified. The RT primer contains a unique molecular identifier (UMI) to reduce PCR bias that could otherwise impact accurate counting of individual mRNA molecules.

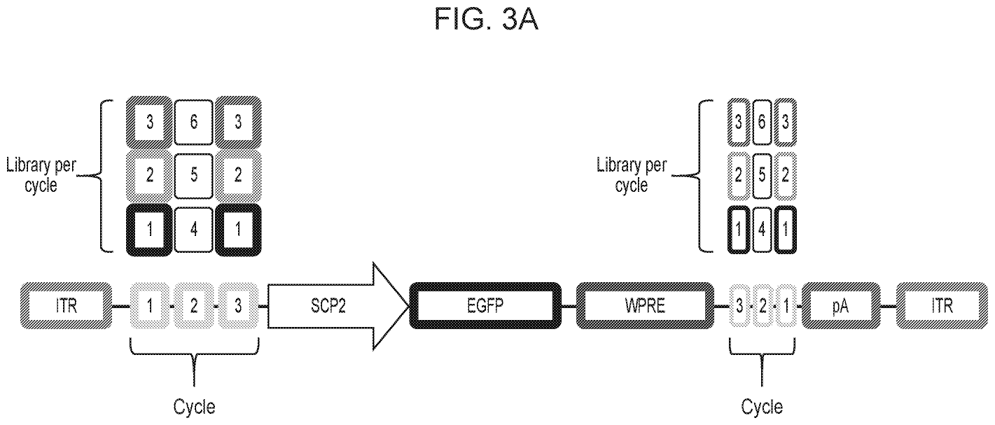

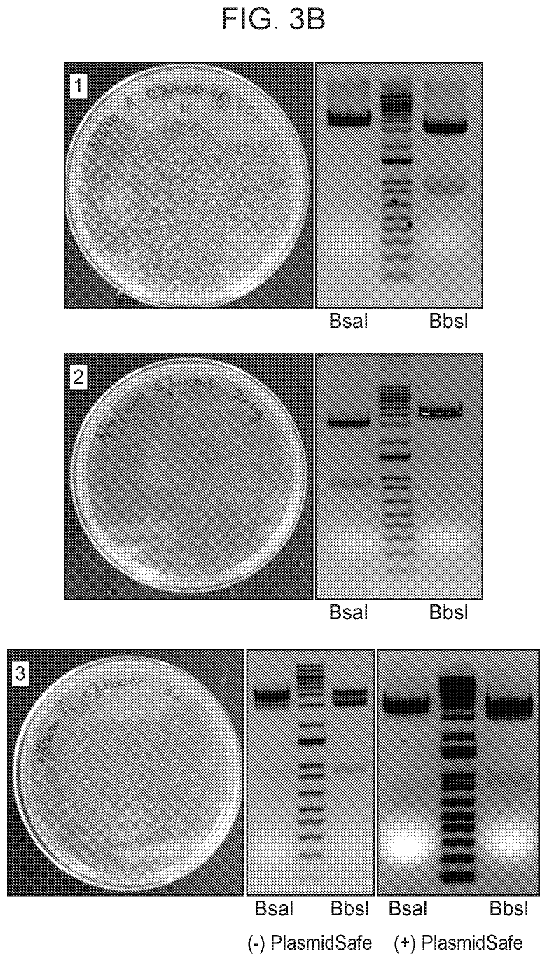

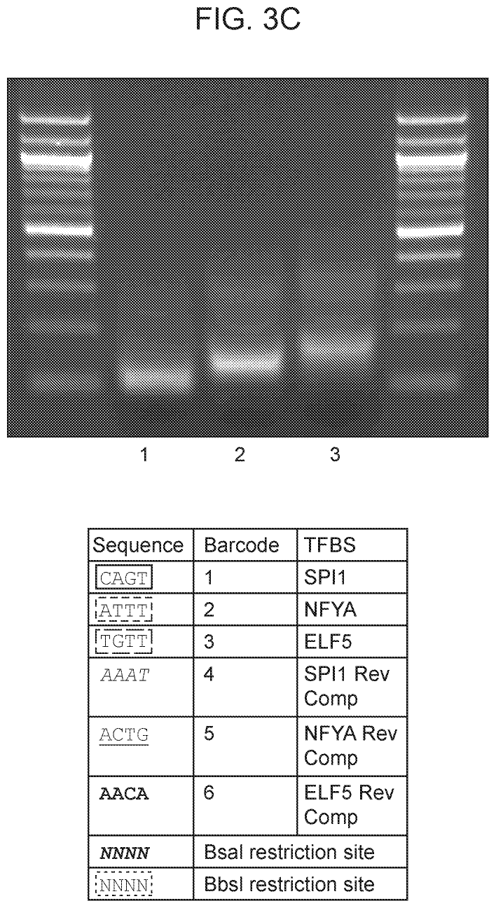

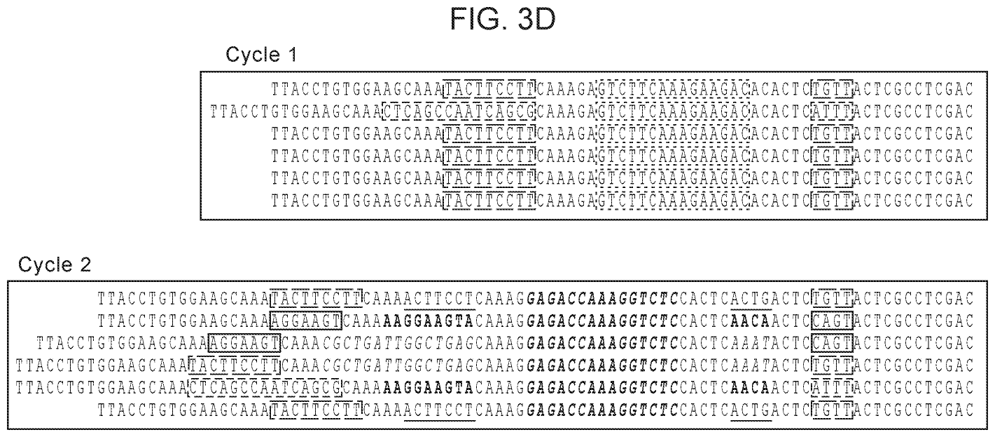

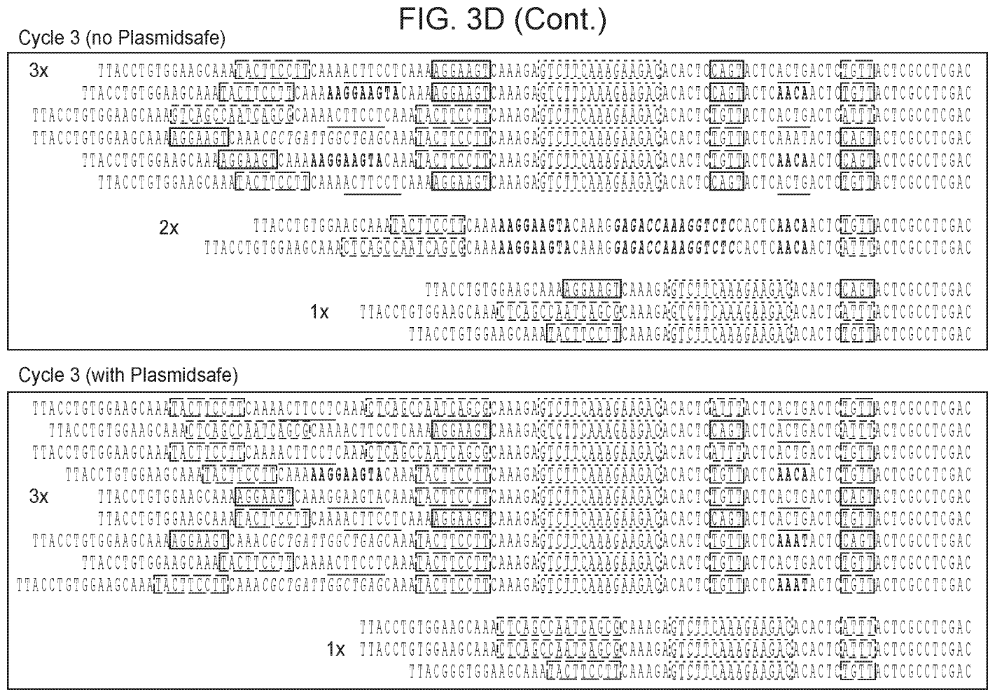

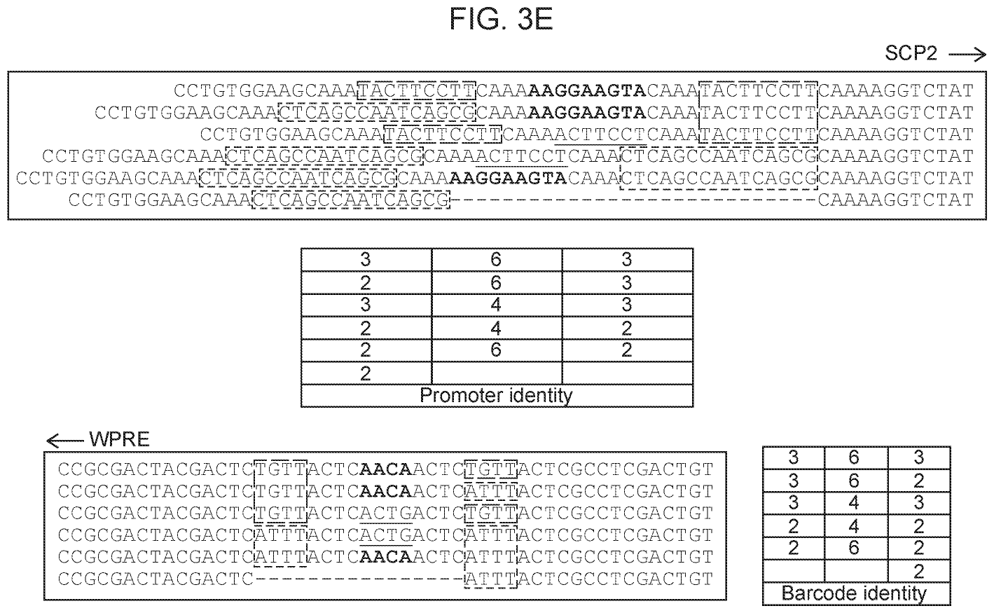

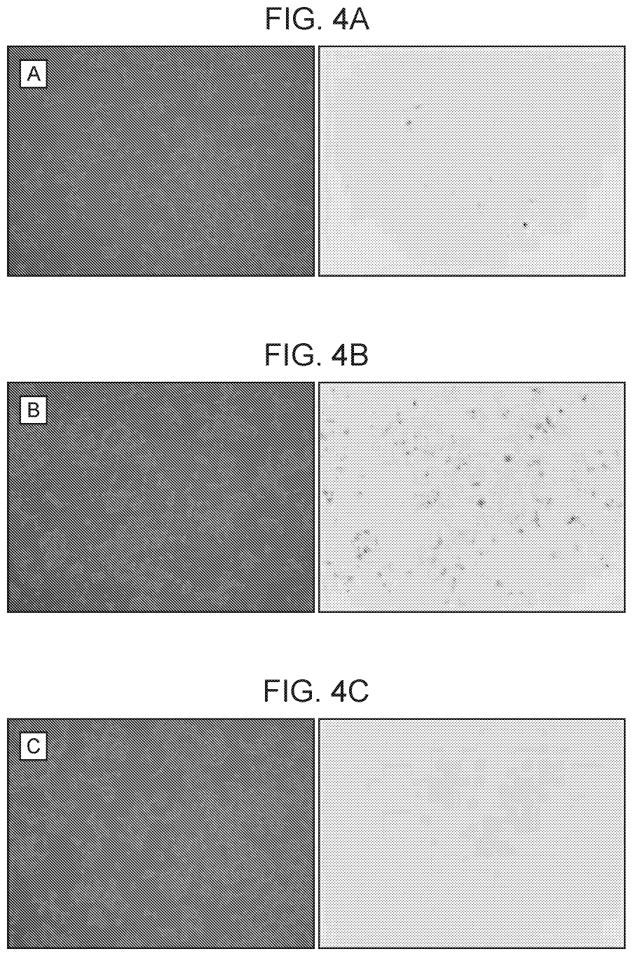

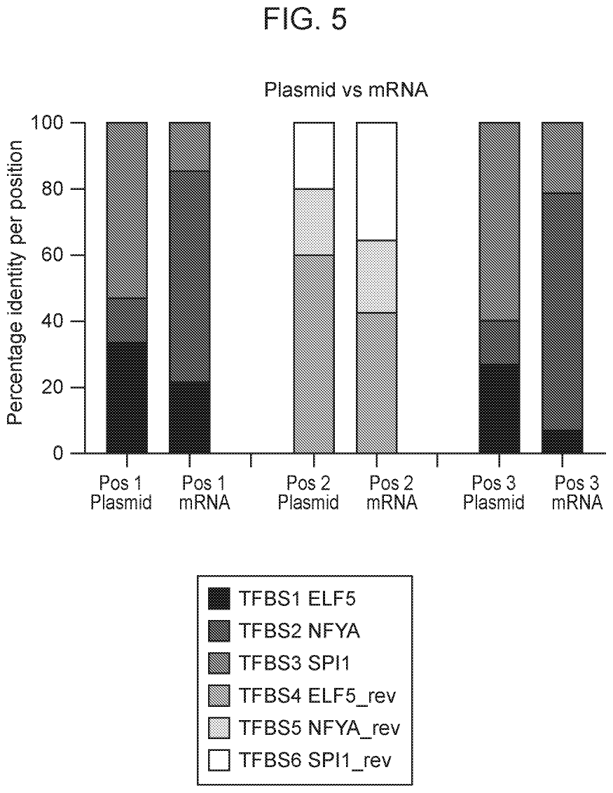

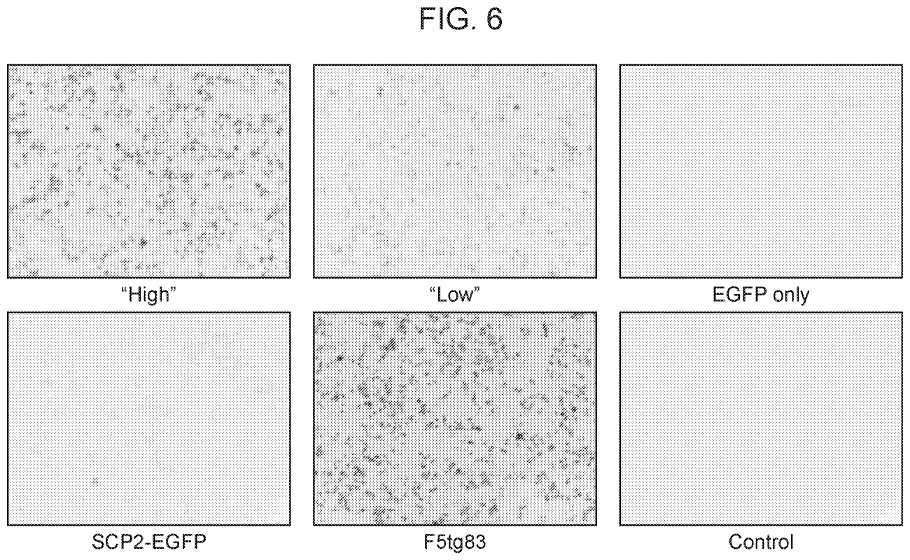

To generate a synthetic promoter library using the ELiPS library generation method, oligo pools from one of the ubiquitous libraries (ELiPS library 2) was used. 3× total TFBS sites and associated barcodes (generation and sequence validation depicted in FIG. 3) were used. The library was used to transfect HEK293T cells, and green fluorescent protein (GFP) signal was observed in a subpopulation of the cells (FIG. 4). RNA was harvested and processed using the targeted RT process (FIG. 3A-3E) to recover the barcodes and subsequently, the promoter sequences from strong and weakly expressing promoters in the 3× library. Based on mRNA prevalence, a ‘high’ expressing plasmid and a ‘low’ expressing plasmid were individually cloned and used to transfect 293T cells. The ratios of particular TFBS motifs found in the plasmid were different than those found in the mRNA, demonstrating a cell-specific expression of each promoter based on the individual TFBS motifs present (FIG. 5). Through a subsequent transfection experiment it was confirmed that the ‘high’ expressing plasmid expressed GFP at levels far higher than that of the ‘low’ plasmid, as hypothesized (FIG. 6).

FIG. 3A-3E. ELiPS library construction test. In this experiment, a library was constructed consisting of three ELiPS cycles. To be able to discern between cycles more easily, the oligo pool of the second cycle differed from the pool used in cycle one and three (FIG. 3A). 50 μl of a total of 500 μl transformed E. coli were plated for each cycle, proving that transformation efficiency does not decrease with successive cycles (FIG. 3B). On each consecutive step, the library was digested with BsaI or BbsI and an enzyme cutting the backbone to address the homogeneity of the library. As is evident in the third cycle, introducing a PlasmidSafe step removes plasmids in which no oligo was ligated in the third cycle. A PCR closely around the ligation site in the library of each cycle showed a size increase consistent with serial ligation of TFBS/BC oligos (FIG. 3C). Sequencing of individual colonies from the plates in (FIG. 3B) proves that each cycle an oligo of the respective pools (BC 1-3 of BC 4-6) was successfully ligated. Again, the PlasmidSafe step removes plasmids of cycle 2 from the library of cycle 3. Sequencing of individual clones following the integration of the transcription cassette shows that each promoter corresponds perfectly with the barcode present in the 3′ UTR sequence (FIG. 3E).

FIG. 4. ELiPS RNA seq proof-of-concept experiment. HEK293T cells were transfected with 2.5 μg of plasmid DNA per 250,000 cells in a 6-well plate. (A) EGFP expression from the 3× TFBS library (B) CMV-EGFP control and (C) no-transfection control. Images taken 18 h post-transfection.

FIG. 5. Differences in percentage identity of TFBS motifs in plasmid vs extracted mRNA. Depending on the choice of TFBS motif, screening in different cell populations will result in stronger expression driven by relative abundance of cell-specific transcription factors (TFs). In position 1 and position 3 of the plasmids, there was a relatively low abundance of the NFYA TFBS motif, but this was highly enriched in recovered mRNA, suggesting that this particular TFBS, and associated TF, is responsible for a larger proportion of expression when compared to the other TFBS.

FIG. 6. GFP expression from individual clones in the 3× TFBS Experiment. Promoters containing highly abundant/enriched mRNA from the plasmid vs mRNA sequencing experiment also exhibited stronger levels of GFP expression in HEK 293T cells via transfection.

To demonstrate the utility of the ELiPS platform to screen large scale promoter libraries, the first pair of ubiquitous libraries (>5×10⁷ members each, with 8× TFBS motifs in each plasmid) was analyzed. HEK 293T cells were transduced at a multiplicity of infection (MOI) of 10 k. RNA was harvested 72 hours later. After targeted RT, barcode recovery, and sequencing through a MiSeq v2 300BP sequencing kit (150PE read protocol), data was processed, and the top 3 hits (determined as a ratio of mRNA count vs count in the plasmid library) from both libraries were individually cloned (Table 2; FIG. 10).

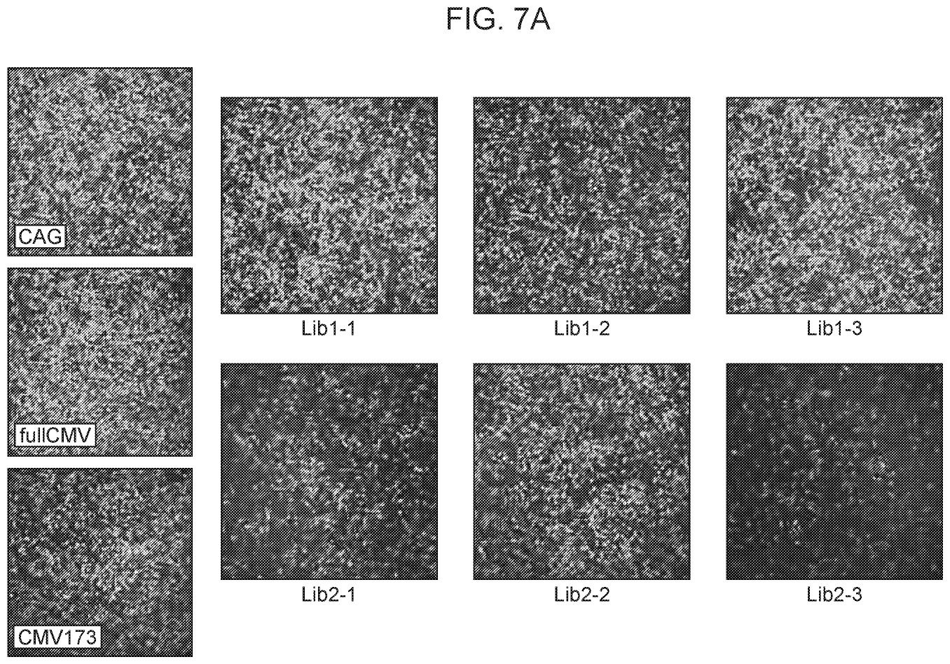

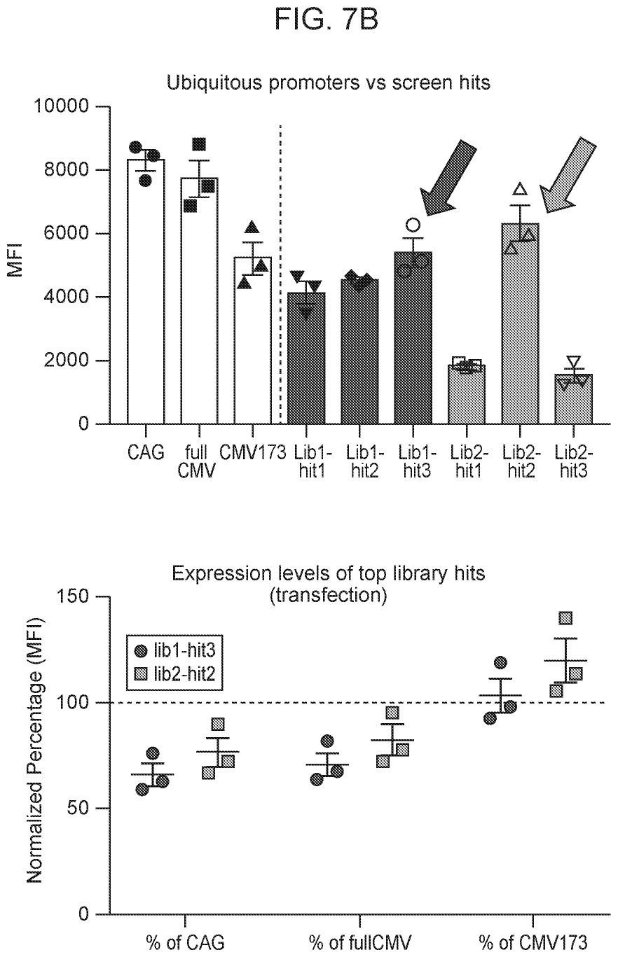

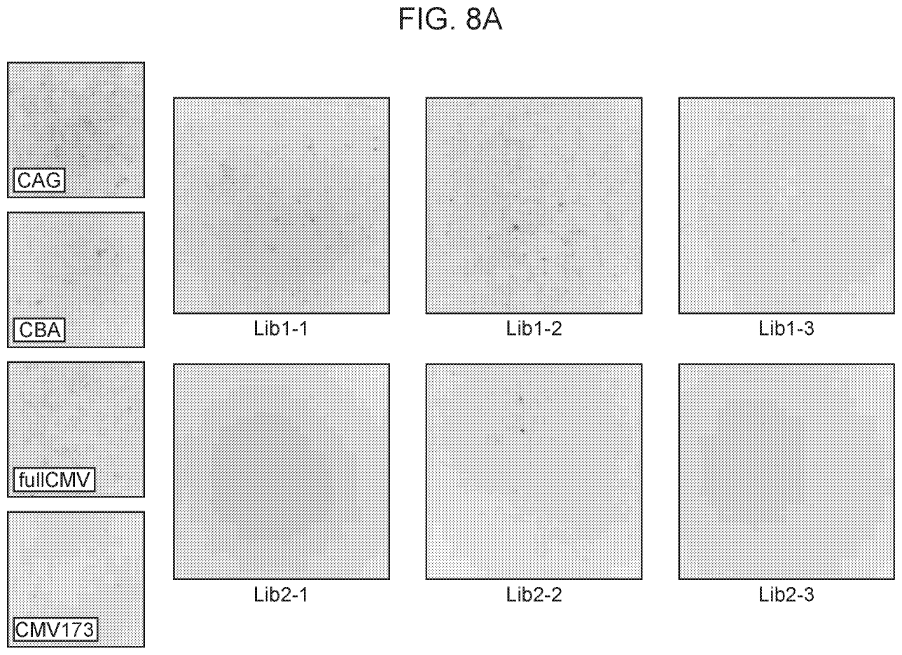

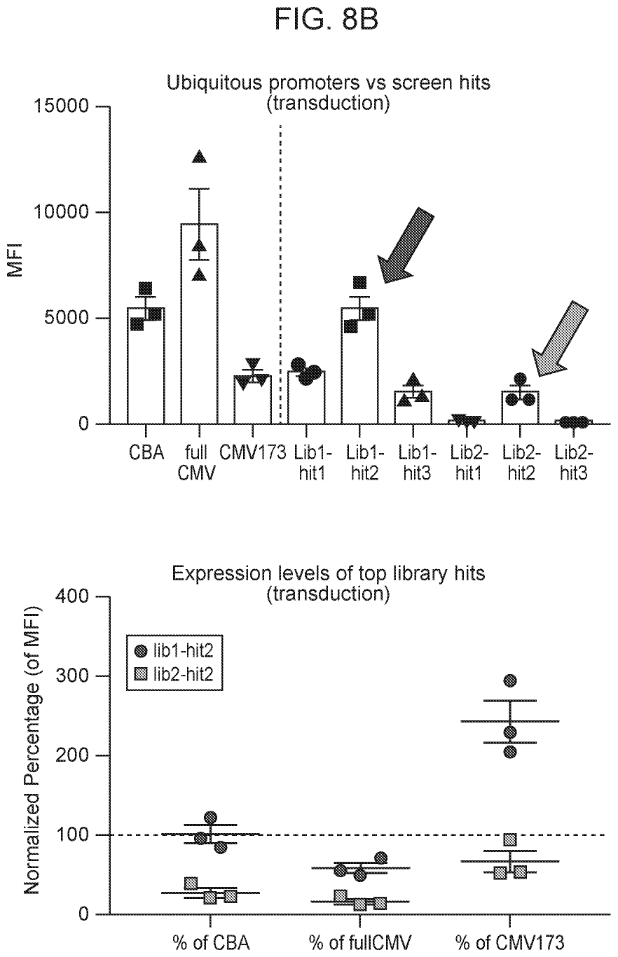

The activity of all 6 promoters set out in Table 2 (FIG. 10) was validated in HEK 293T cells through transfection (FIG. 7) and transduction (FIG. 8). These 6 promoters demonstrated high levels of activity—in transfection tests, one in particular (Lib2-hit2, denoted as “EL2T.1”, 218 bp) has 76% of the activity of the CAG promoter (1664 bp) and 82% of the activity of the CMV promoter (808 bp)—via flow cytometry, MFI_{CAG} 8570±611, MFI_{CMV} 7985±1128, MFI_{EL2T.1} 6583±1118, at a 95% CI. The expression level of EL2T.1 is also not statistically significantly different from that of CMV (p=0.159, two-tailed Student's t-test, unequal variance). In transduction tests, one of the hits (lib1-hit2, denoted as “EL1T.1”, 193 bp) has 100% the activity of the CBA promoter (934 bp) and 58% the activity of the CMV promoter (808 bp)—via flow cytometry, MFI_{CBA} 5452±989, MFI_{CMV} 9434±3272, MFI_{EL1T.1} 5481±1189, at a 95% CI. The expression level of EL1T.1 is also not statistically significantly different from that of CMV (p=0.113, two-tailed Student's t-test, unequal variance).

FIG. 7. Top promoters from ubiquitous ELiPS libraries—Transfection. The top three promoters from both ubiquitous libraries were individually cloned used to transfect 250 k HEK 293T cells (375 ng total DNA, at 500 ng*cm⁻¹ using PEI.). 24 hrs post-transfection, cells were assessed for GFP signal (correlating to promoter strength) via flow cytometry. Background signal from untransfected cells was subtracted; the right panel denotes promoter strength as a percentage of the constitutive strong promoters. Lib2-hit2 has been internally termed “EL2T.1”.

FIG. 8. Top promoters from ubiquitous ELiPS libraries—Transduction. The top three promoters from both ubiquitous libraries were individually cloned used to transduce HEK 293T cells at an MOI of 20 k with the A101 capsid. 96 hrs post-transduction, cells were assessed for GFP signal (correlating to promoter strength) via flow cytometry. Background signal from untransfected cells was subtracted; the right panel denotes promoter strength as a percentage of the constitutive strong promoters. Brightness has been increased through postprocessing in the images. Lib1-hit2 has been internally termed “ELIT.1”.

Table 2 (FIG. 10). Top promoters from ubiquitous ELiPS libraries. TFBS identity and location of each motif comprising the top six ubiquitous promoters. BC denotes barcode location in the promoter, and a “_rev” indication denotes the binding site for that particular TF was in reverse (3′-5′) orientation. Between each TBFS motif, there is an ‘ACTC’ sequence used as a spacer. In each promoter, the SCP2 sequence is underlined.

### Example 2

Methods of further increasing the strength of ELiPS promoters were explored based on their unique architecture. Like endogenous mammalian promoters, ELiPS promoters contain an enhancer region (comprised of cis-regulatory elements, CREs) upstream of a core promoter. However, the enhancer region is drastically shorter than that of a typical endogenous promoter (˜120 bp versus hundreds or thousands of bp long), and the local concentration of transcription factor binding sites is much higher (separated by only 4 bp versus tens or hundreds of bp). Activity of a promoter has been correlated with binding interactions of TFs with their corresponding TFBSs—the more binding interactions, even if transient, results in higher levels of promoter activity. Even though the enhancer element is so short, the 8 TFBS motifs in the ELiPS promoters allow for an increased likelihood of TF interactions—to take further advantage of this enhancer architecture, the segment containing these TFBS binding sites was doubled or tripled. Additionally, it was sought to increase the strength of the ELiPS promoters through the addition of intronic elements, which has been shown to act through orthogonal mechanisms to the enhancer to increase transcript stability and mRNA export from the nucleus.

Constructs were individually cloned representing these variations (FIG. 11) into the top hit from each library in the 293T screen (Lib1-hit2, denoted as 007 and Lib2-hit2, denoted as 010). The specific sequences and sizes of each promoter construct is listed in Table 3 (FIG. 13). These promoters were then compared against strong ubiquitous viral control promoters and assessed for their ability to drive eGFP expression both through plasmid transfection and AAV-mediated transduction.

FIG. 11 shows that the ELiPS synthetic enhancer elements (comprised of ˜8× TFBS separated by 4 bp spacers) can be repeated in tandem, either alone with SCP2 or in combination with an intron (in this case, the SV40 intron) for significant increases in promoter strength. A base ELiPS promoter is ˜200 bp, with the triple enhancer versions or double enhancer+SV40 intron versions being up to ˜450 bp depending on the exact enhancer sequence.

Table 3 (FIG. 13) includes the sequence identity of variants of the top two 293T promoter hits. Enhancer elements were repeated in tandem and in combination with the SV40 intron. In each promoter, the SCP2 sequence is underlined.

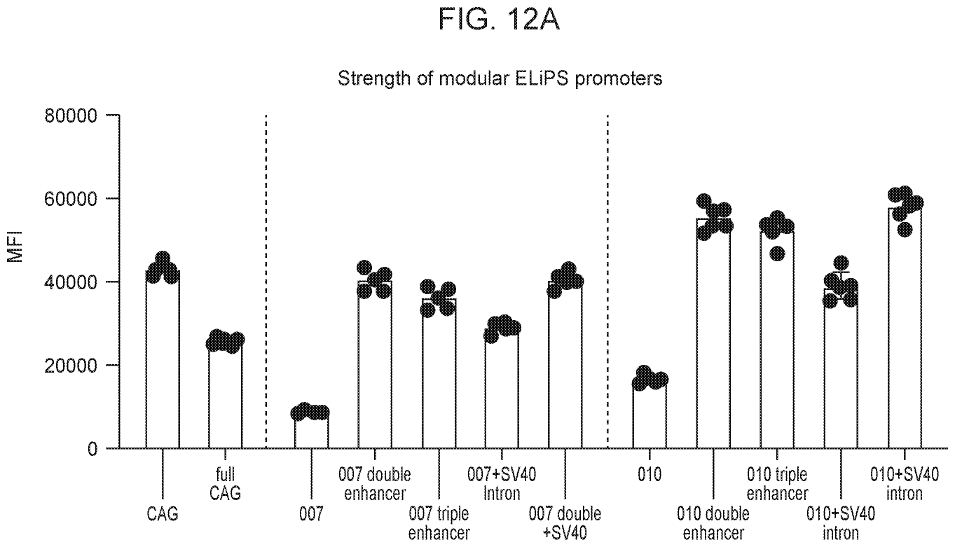

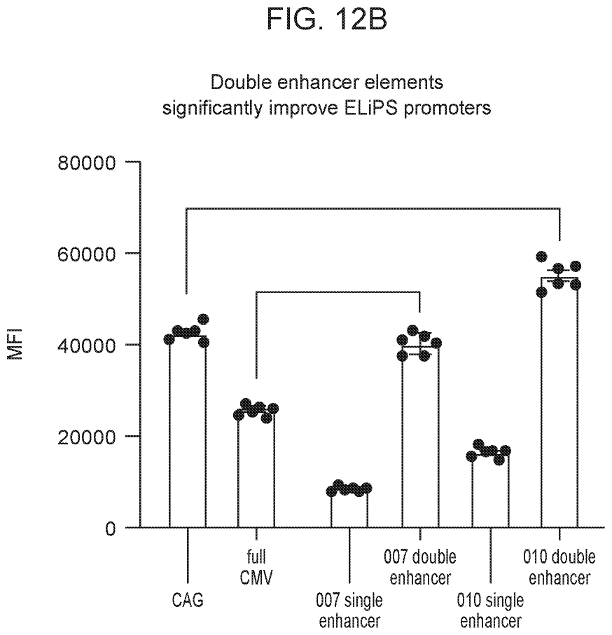

In plasmid transduction, the addition of either a double or triple enhancer element to each promoter significantly increased eGFP expression strength (FIG. 12A-12B). The largest boost to activity came from the addition of a single extra enhancer unit, with the expression level of the triple enhancer promoters being slightly lower than that of the double enhancer. The addition of the SV40 intronic element significantly increased expression levels over the base forms of the promoters (FIG. 12A-12B). A second enhancer element in tandem with the SV40 intron also significantly increased strength versus having a single enhancer and SV40 intron, though this boost was largely driven by the additional enhancer element.

FIG. 12A-12B shows that the addition of tandem arrays of the ELiPS enhancer portion, in combination with the SV40 intron, can significantly improve the expression levels of the promoters with only a modest increase in length. **** p<0.0001, two-tailed Welch's t-test, unequal variance.

Notably, the lib2-hit2 double enhancer promoter (010-double enhancer, 355 bp) was not only significantly stronger than the full-length CMV promoter but also the CAG promoter, while being less than 25% of the size. This promoter appeared capable of driving expression strength in 293T cells via plasmid transfection at levels significantly higher than any other promoter reported in the literature.

With this information about the significant improvements made by tandem enhancer elements and the SV40 intron with the ELiPS promoter architecture, it was concluded that these sequences and all tandem enhancer promoters modeled on the base forms of the ELiPS promoters, either alone or in combination with the SV40 intron, may be employed as promoters for protected use in transfection and transduction-based gene expression platforms.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for generating a synthetic transcriptional promoter that is functional in a eukaryotic cell, the method comprising:
A) introducing an expression vector into a eukaryotic cell, wherein the expression vector comprises:
a) a synthetic transcriptional promoter comprising:
i) a first transcription factor binding site (TFBS) comprising an upstream enhancer element of from 4 to 20 base pairs (bp) in length;
ii) at least a second TFBS, wherein the at least a second TFBS comprises an upstream enhancer element of from 4 to 20 bp and has a nucleotide sequence that is the same or different from the first TFBS; and
iii) a core promoter comprising:
a TATA box;
an initiator element;
an RNA Polymerase II binding site; and
a transcription start site; and
b) a nucleotide sequence encoding a reporter polypeptide, wherein the nucleotide sequence encoding the reporter polypeptide is operably linked to the synthetic transcriptional promoter; and
B) detecting expression of the reporter polypeptide, wherein expression of the reporter polypeptide in the eukaryotic cell indicates that the synthetic transcriptional promoter that is functional in the eukaryotic cell.

2. The method of claim 1, wherein the expression vector comprises from 2 to 30 TFBS.

3. The method of claim 2, wherein the expression vector comprises a nucleic acid barcode that identifies the combination of the from 2 to 30 TFBS.

4. The method of claim 1, wherein the synthetic transcriptional promoter has a length of no more than about 700 bp.

5.-11. (canceled)

12. A library of expression vectors comprising a plurality of members comprising:
a) a synthetic transcriptional promoter comprising:
i) a first transcription factor binding site (TFBS) comprising an upstream enhancer element of from 4 to 20 base pairs (bp) in length;
ii) at least a second TFBS, wherein the at least a second TFBS comprises an upstream enhancer element of from 4 to 20 bp and has a nucleotide sequence that is the same or different from the first TFBS; and
iii) a core promoter comprising:
a TATA box;
an initiator element;
an RNA Polymerase II binding site; and
a transcription start site; and
b) a nucleotide sequence encoding a reporter polypeptide, wherein the nucleotide sequence encoding the reporter polypeptide is operably linked to the synthetic transcriptional promoter.

13.-19. (canceled)

20. The library of claim 12, wherein the library comprises from 10² to 10¹¹ members.

21. A functional synthetic transcriptional promoter comprising a nucleotide sequence having at least 90% nucleotide sequence identity to any one of the nucleotide sequences depicted in FIG. 10 or FIG. 13.

22. The functional synthetic transcriptional promoter of claim 21, comprising a nucleotide sequence having at least 95%, at least 98%, at least 99%, or 100%, nucleotide sequence identity to the promoter sequence identified as EL1T.1 in FIG. 10.

23. The functional synthetic transcriptional promoter of claim 21, comprising a nucleotide sequence having at least 95%, at least 98%, at least 99%, or 100%, nucleotide sequence identity to the promoter sequence identified as EL2T.1 in FIG. 10.

24. A recombinant expression vector comprising the synthetic transcriptional promoter of claim 21.

25. The recombinant expression vector of claim 24, wherein the synthetic transcriptional promoter is operably linked to a nucleotide sequence encoding a polypeptide of interest.

26. The recombinant expression vector of claim 24, wherein the vector is an adeno-associated virus (AAV) vector.

27. The recombinant expression vector of claim 24, wherein the vector is a lentivirus vector or an adenovirus vector.

28. A composition comprising the recombinant expression vector of claim 24.

29. The composition of claim 28, comprising a nanoparticle, a lipid, or a liposome.

30. A eukaryotic cell genetically modified with:
a) the functional synthetic transcriptional promoter of claim 21; or
b) the recombinant expression vector of claim 24.

31. The eukaryotic cell of claim 30, wherein the cell is a mammalian cell.

32. A method of generating a recombinant expression vector comprising a synthetic transcriptional promoter, the method comprising:
a) introducing into an expression vector a first nucleic acid comprising: i) a first transcription factor binding site (TFBS) comprising an upstream enhancer element of from 4 to 20 base pairs (bp) in length; ii) a first restriction enzyme recognition site; and iii) a first barcode that identifies the first TFBS,
wherein the first restriction enzyme site is not present elsewhere in the expression vector,
wherein said introducing results in a first modified expression vector;
b) cleaving the first modified expression vector with a restriction enzyme that cleaves the first restriction enzyme recognition site, generating a first linear modified expression vector;
c) ligating to the first linear modified expression vector a second nucleic acid comprising: i) a second TFBS comprising an upstream enhancer element of from 4 to 20 base pairs (bp) in length; ii) a second restriction enzyme recognition site; and iii) a second barcode, wherein:
the second TFBS has the same nucleotide sequence or a different in nucleotide sequence from the first TFBS,
the second restriction enzyme site is not present elsewhere in the expression vector and is different from the first restriction enzyme site, and
the second barcode identifies the second TFBS;
wherein said ligating results in a second modified expression vector;
d) cleaving the second modified expression vector with a restriction enzyme that cleaves the second restriction enzyme recognition site, resulting in a second linear modified expression vector; and
e) ligating to second linear modified expression vector a nucleic acid comprising: i) a core promoter; and ii) a nucleotide sequence encoding a reporter polypeptide,
wherein said ligating results in a recombinant expression vector comprising: i) a synthetic transcriptional promoter comprising at least two TFBSs and the core promoter; and ii) a composite barcode comprising the two barcodes, wherein the composite barcode identifies the two TFBSs, wherein the composite barcode is 3′ of the nucleotide sequence encoding the reporter polypeptide.

33. The method of claim 32, further comprising repeating steps (a) through (c) to insert at least a third nucleic acid comprising: i) a third TFBS comprising an upstream enhancer element of from 4 to 20 base pairs (bp) in length; ii) the first restriction enzyme recognition site; and iii) a third barcode,
thereby generating a recombinant expression vector comprising: i) a synthetic transcriptional promoter comprising at least three TFBSs and the core promoter; and ii) a composite barcode comprising the three barcodes, wherein the composite barcode identifies the three TFBSs.

34.-41. (canceled)

42. A method of producing a library of recombinant expression vectors, each comprising a different synthetic transcriptional promoter, the method comprising carrying out the method of claim 32 with a plurality of expression vectors, to generate a library of recombinant expression vectors, each comprising a different synthetic transcriptional promoter, each with a unique composite barcode.

43.-44. (canceled)
